(19) United States
(12) Patent Application Publication
(10) Pub. No.: US 2026/0232710 A1
(43) Pub. Date: Aug. 13, 2026

(54) AGENTS, COMPOSITIONS, AND METHODS FOR SUPPORTING BEHAVIOURAL DEVELOPMENT

(71) Applicant: SOCIÉTÉ DES PRODUITS NESTLÉ S.A., Vevey (CH)

(72) Inventors: Purva RAJHANS, Lausanne (CH); Nora SCHNEIDER, Les Monts-de-Corsier (CH); Jonas HAUSER, Lausanne (CH); Hongtu ZHU, Chapel Hill, NC (US); Tengfei LI, Chapel Hill, NC (US); Seoyoon CHO, Chapel Hill, NC (US); Weili LIN, Chapel Hill, NC (US)

(21) Appl. No.: 19/151,909

(22) PCT Filed: Jan. 31, 2024
(86) PCT No.: PCT/EP2024/052415

§ 371 Date: Jul. 29, 2025

Related U.S. Application Data
(60) Provisional application No. 63/462,621, filed on Apr. 28, 2023.

(30) Foreign Application Priority Data
Jan. 31, 2023 (EP) 23154263.0

(51) Int. Cl. A61K 31/702; A61K 31/7016; A61P 25/28

(52) U.S. Cl. CPC A61K 31/702; A61K 31/7016; A61P 25/28

(57) ABSTRACT

The present invention relates to one or more human milk oligosaccharides (HMOs) for use in supporting behavioural development in a subject, wherein the one or more HMOs comprise or consist of: (a) lacto-N-difucohexaose I (LNDFHI); and/or (b) 2′-fucosyllactose (2′FL), 3-fucosyllactose (3FL), lactodifucotetraose (LDFT), lacto-N-fucopentaose II (LNFPII), lacto-N-fucopentaose I (LNFP-I), lacto-N-fucopentaose III (LNFP-III), lacto-N-difucohexaose I (LND-I), monofucosyl-lacto-N-hexaose III (MFLNH-III), lacto-N-fucopentaose V (LNFP-V), lacto-N-neodifucohexaose (LNnDFH) and lacto-N-fucopentaose VI (LNFP-VI).

### FIELD OF THE INVENTION

The present invention relates to agents, compositions and methods for supporting behavioural development. The present invention further relates to agents, compositions and methods for improving social-emotional skills, reducing negative affectivity, and preventing an anxiety disorder and/or mood disorder.

### BACKGROUND TO THE INVENTION

Social-emotional skills develop together in infancy and throughout childhood and include behaviours that represent children's emotional growth and their ability to successfully interact with people. Social-emotional development includes the development of emotional skills and competencies, including mood. Emotional development may refer to the ability to effectively regulate emotions to accomplish one's goal.

It is believed that lack of social-emotional skills can lead to poor outcomes in a child's behavioural development, possibly leading to aggressive or disruptive behaviours, loneliness, anxiety and depression. Many anxiety disorders have their onset in early childhood (see e.g. Clark C., et al., 2007. Arch Gen Psychatry. 64(6), pp. 668-678 and Mian N., et al., 2012. J. Anxiety Disord. 26(1), pp 102-110).

Whilst studies have indicated an association between single compounds found in human breast milk and behavioural development, the relationship between nutrition during early life and its impact on the behavioural development of infants, toddlers and/or young children remains unclear (see e.g. Wachs, T. D., 2000. International Journal of Behavioral Development, 24(4), pp. 435-441).

Thus, there is a demand for new nutritional interventions to promote behavioural development and/or improve social-emotional skills, for example in infants, toddlers or young infants. There is a need to deliver such benefit in a manner that does not induce side effects and/or in a manner that is easy to deliver, and well accepted by the parents or health care practitioners. There is also a need to deliver such benefits in a manner that keeps the cost of such delivery reasonable and affordable by most.

### SUMMARY OF THE INVENTION

The present inventors have surprisingly found that specific human milk oligosaccharides (HMOs) are associated with a reduction in negative emotions in human milk-fed infants. The HMOs identified by the inventors have not previously been reported to be associated with social-emotional outcomes.

A reduction of negative emotions may be beneficial for behavioural development, as infancy is a pivotal age for the social-emotional milestones such as the experience of fear, the emergence of separation anxiety with primary care-giver, the visual recognition of familiar people, and letting parents know when happy versus when upset.

In one aspect, the present invention provides one or more human milk oligosaccharides (HMOs) for use in supporting behavioural development in a subject.

In one aspect, the present invention provides one or more human milk oligosaccharides (HMOs) for use in supporting behavioural development in a subject, wherein the one or more HMOs comprise or consist of: (a) lacto-N-difucohexaose I (LNDFHI); and/or (b) 2′-fucosyllactose (2′FL), 3-fucosyllactose (3FL), lactodifucotetraose (LDFT), and lacto-N-fucopentaose II (LNFPII), LNFP-I (lacto-N-fucopentaose I (LNFP-I), lacto-N-fucopentaose III (LNFP-III), lacto-N-difucohexaose I (LND-I), monofucosyl-lacto-N-hexaose III (MFLNH-III), lacto-N-fucopentaose V (LNFP-V), lacto-N-neodifucohexaose (LNnDFH) and, lacto-N-fucopentaose VI (LNFP-VI).

In another aspect, the present invention provides use of one or more human milk oligosaccharides (HMOs) for supporting behavioural development in a subject.

In another aspect, the present invention provides a method for supporting behavioural development in a subject, comprising administering to the subject one or more human milk oligosaccharides (HMOs).

In another aspect, the present invention provides use of one or more HMOs for improving social-emotional skills, wherein the one or more HMOs comprise or consist of: (a) lacto-N-difucohexaose I (LNDFHI); and/or (b) 2′-fucosyllactose (2′FL), 3-fucosyllactose (3FL), lactodifucotetraose (LDFT), lacto-N-fucopentaose II (LNFPII), lacto-N-fucopentaose I (LNFP-1), lacto-N-fucopentaose III (LNFP-III), lacto-N-difucohexaose I (LND-I), monofucosyl-lacto-N-hexaose III (MFLNH-III), lacto-N-fucopentaose V (LNFP-V), lacto-N-neodifucohexaose (LNnDFH) and lacto-N-fucopentaose VI (LNFP-VI).

In another aspect, the present invention provides use of one or more HMOs for reducing negative affectivity, wherein the one or more HMOs comprise or consist of: (a) lacto-N-difucohexaose I (LNDFHI); and/or (b) 2′-fucosyllactose (2′FL), 3-fucosyllactose (3FL), lactodifucotetraose (LDFT), lacto-N-fucopentaose II (LNFPII), lacto-N-fucopentaose I (LNFP-1), lacto-N-fucopentaose III (LNFP-III), lacto-N-difucohexaose I (LND-I), monofucosyl-lacto-N-hexaose III (MFLNH-III), lacto-N-fucopentaose V (LNFP-V), lacto-N-neodifucohexaose (LNnDFH) and lacto-N-fucopentaose VI (LNFP-VI).

Any suitable HMOs described herein may be used. Suitably, the one or more HMOs comprise or consist of a combination of fucosylated HMOs. Suitably, the one or more HMOs comprise or consist of: (a) Lacto-N-difucohexaose I (LNDFHI); and/or (b) 2′-fucosyllactose (2′FL), 3-fucosyllactose (3FL), lactodifucotetraose (LDFT), lacto-N-fucopentaose II (LNFPII), lacto-N-fucopentaose I (LNFP-I), lacto-N-fucopentaose III (LNFP-III), lacto-N-difucohexaose I (LND-I), monofucosyl-lacto-N-hexaose III (MFLNH-III), lacto-N-fucopentaose V (LNFP-V), lacto-N-neodifucohexaose (LNnDFH) and lacto-N-fucopentaose VI (LNFP-VI). In some embodiments, the one or more HMOs comprise or consist of LNDFHI. In some embodiments, the one or more HMOs comprise or consist of 2′FL, 3FL, LDFT, LNFPII, LNFP-I, LNFP-III, LND-I, MFLNH-III, LNFP-V, LNnDFH and LNFP-VI.

The one or more HMOs may be administered by any suitable means and in any suitable amounts. Suitably, the one or more HMOs are administered by oral administration. Suitably, the one or more HMOs are administered separately, simultaneously or sequentially. In preferred embodiments, the one or more HMOs are administered simultaneously. Suitably, the one or more HMOs are administered to the subject in a total amount of from about 0.5 g/day to about 10 g/day. Suitably, a combination of fucosylated HMOs are administered to the subject in a total amount of from about 0.5 g/day to about 10 g/day. Suitably, LNDFHI is administered to the subject in an amount of from about 0.1 g/day to about 5 g/day. Suitably, 2′FL is administered to the subject in an amount of from about 0.1 g/day to about 5 g/day; 3FL is administered to the subject in an amount of from about 0.1 g/day to about 5 g/day; LDFT is administered to the subject in an amount of from about 0.1 g/day to about 5 g/day; LNFPII is administered to the subject in an amount of from about 0.1 g/day to about 5 g/day. LNFP-I is administered to the subject in an amount of from about 0.1 g/day to about 5 g/day; LNFP-III is administered to the subject in an amount of from about 0.1 g/day to about 5 g/day; LND-I is administered to the subject in an amount of from about 0.1 g/day to about 5 g/day; MFLNH-III is administered to the subject in an amount of from about 0.1 g/day to about 5 g/day; LNFP-V is administered to the subject in an amount of from about 0.1 g/day to about 5 g/day; LNnDFH is administered to the subject in an amount of from about 0.1 g/day to about 5 g/day; and/or LNFP-VI is administered to the subject in an amount of from about 0.1 g/day to about 5 g/day.

The one or more HMOs may be provided in any suitable form. Suitably, the one or more HMOs are in the form of a nutritional composition. In some embodiments, the nutritional composition is an infant formula, a follow-on formula, or a growing-up milk. In preferred embodiments, the nutritional composition is an infant formula. Suitably, the starter infant formula, follow-on formula, and growing-up milk each comprise from about 60 kcal/100 mL to about 80 kcal/100 mL, protein in an amount of from about 1.5 g/100 kcal to about 2.5 g/100 kcal, carbohydrate in an amount of from about 8 g/100 kcal to about 15 g/100 kcal, and lipids in an amount of from about 3 g/100 kcal to about 8 g/100 kcal.

The nutritional composition may comprise the one or more HMOs in any suitable amount. Suitably, the nutritional composition comprises the one or more HMOs in a total amount of from about 0.5 g/L to about 10 g/L. Suitably, the nutritional composition comprises a combination of fucosylated HMOs in a total amount of from about 0.5 g/L to about 10 g/L. Suitably, the nutritional composition comprises LNDFHI in an amount of from about 0.2 g/L to about 2.0 g/L. Suitably, the nutritional composition comprises 2′FL in an amount of from about 0.5 g/L to about 3.0 g/L; 3FL in an amount of from about 0.2 g/L to about 2.0 g/L; LDFT in an amount of from about 0.05 g/L to about 0.5 g/L; LNFPII in an amount of from about 0.05 g/L to about 0.5 g/L; LNFP-1 in an amount of from about 0.2 g/L to about 2.0 g/L; LNFP-11l in an amount of from about 0.2 g/L to about 2.0 g/L; LND-I in an amount of from about 0.2 g/L to about 2.0 g/L; MFLNH-III in an amount of from about 0.2 g/L to about 2.0 g/L; LNFP-V in an amount of from about 0.2 g/L to about 2.0 g/L; LNnDFH in an amount of from about 0.2 g/L to about 2.0 g/L; and/or LNFP-VI in an amount of from about 0.2 g/L to about 2.0 g/L.

The subject may be any suitable subject. The subject may be a human subject. In some embodiments, the subject is an infant, a toddler or a young child. In preferred embodiments, the subject is an infant. In some embodiments, the subject is aged about 12 months or younger, about 11 months or younger, about 10 months or younger, or about 9 months or younger.

The one or more HMOs may improve the subject's social-emotional skills. The one or more HMOs may reduce the subject's negative affectivity. The subject's negative affectivity may be determined by an Infant Behavior Questionnaire (IBQ), for example the IBQ-Revised (IBQ-R).

### DESCRIPTION OF DRAWINGS

FIG. 1—Association of LNDFHI in breast milk at 2 months with reduction in negative emotions in infants at 9 months

Regression model approach was used to determine association between LNDFHI in breast milk and negative affectivity constructed out of several Infant Behaviour Questionnaire-Revised (IBQR) scales, N=195, p=0.0079.

FIG. 2—Association of 2′FL, 3FL, LDFT and LNFP II in breast milk at 2 months with reduction in negative emotions at 9 months

Regression model approach was used to determine association between 2′-fucosyllactose (2′FL), 3-fucosyllactose (3FL), lactodifucotetraose (LDFT) and lacto-N-fucopentaose II (LNFP II) in breast milk and negative affectivity constructed out of several Infant Behaviour Questionnaire-Revised (IBQR) scales, N=195, p=0.026.

FIG. 3—Association of 3FL in breast milk with (A) gross motor skills and (B) visual reception Scatter plot of the subject Mullen Scales of Early Learning (MSEL) subscale score for gross motor (A) or visual reception (B) with breastmilk 3FL levels. A liner mixed model approach was used and there was a significant association between 3FL in breast milk and gross motor skills and visual reception MSEL subscale scores of infant (N=170, p=0.041 and 0.027, respectively). In order to remove the potential confounding effects from age, age effect was removed from the HMOs using smooth spline regression prior to conducting association

FIG. 4 A and B—Association of LNDFH-I, 2′FL, 3FL, LDFT, LNFPII, LNFP-I, LNFP-III, LND-I, MFLNH-III, LNFP-V, LNnDFH and LNFP-VI in breast milk at 2 months and 3 months with reduction in negative emotions at 9 months

Regression model approach was used to determine association between lacto-N-difucohexaose I (LNDFHI), 2′-fucosyllactose (2′FL), 3-fucosyllactose (3FL), lactodifucotetraose (LDFT), and lacto-N-fucopentaose II (LNFPII), LNFP-I (lacto-N-fucopentaose I (LNFP-I), lacto-N-fucopentaose III (LNFP-III), lacto-N-difucohexaose I (LND-I), monofucosyl-lacto-N-hexaose III (MFLNH-III), lacto-N-fucopentaose V (LNFP-V), lacto-N-neodifucohexaose (LNnDFH) and lacto-N-fucopentaose VI (LNFP-VI) in breast milk and negative affectivity constructed out of several Infant Behaviour Questionnaire-Revised (IBQR) scales at 2 months, N=195, p=0.0056, and 3 months N=195, p=0.0037 respectively.

### DETAILED DESCRIPTION

Various preferred features and embodiments of the present invention will now be described by way of non-limiting examples. The skilled person will understand that they can combine all features of the invention disclosed herein without departing from the scope of the invention as disclosed.

Any reference to prior art documents in this specification is not to be considered an admission that such prior art is widely known or forms part of the common general knowledge in the field. All publications mentioned in the specification are herein incorporated by reference.

As used in this specification, the words “comprises”, “comprising”, and similar words, are not to be interpreted in an exclusive or exhaustive sense. In other words, they are intended to mean “including, but not limited to”. The terms “comprises”, “comprising”, and similar words also include the term “consisting of”.

The practice of the present invention will employ, unless otherwise indicated, conventional techniques which are within the capabilities of one of ordinary skill in the art. Such techniques are explained in the literature. Unless defined otherwise, all technical and scientific terms used herein have the same meaning as commonly understood by one of ordinary skill in the art.

Numeric ranges are inclusive of the numbers defining the range and all percentages disclosed herein are on a w/w basis, unless stated otherwise. As used herein the term “about” means approximately, in the region of, roughly, or around. When the term “about” is used in conjunction with a numerical value or range, it modifies that value or range by extending the boundaries above and below the numerical value(s) set forth. In general, the terms “about” and “approximately” are used herein to modify a numerical value(s) above and below the stated value(s) by 10%.

### Human Milk Oligosaccharides (HMOs)

The present invention relates to the use of one or more human milk oligosaccharides (HMOs) to promote behavioural development, improve social-emotional skills, reduce negative affectivity, and/or prevent an anxiety disorder and/or mood disorder. In some embodiments, the one or more HMOs are used in a therapeutic method.

The one or more HMOs may be administered by any suitable route and in any suitable form. Suitably, the one or more HMOs are administered by oral and/or enteral administration. In preferred embodiments, the one or more HMOs are administered by oral administration. The one or more HMOs may be administered separately, simultaneously or sequentially. In preferred embodiments, the one or more HMOs are administered simultaneously.

Many different kinds of HMOs are found in human milk and are typically based on a combination of glucose, galactose, sialic acid (N-acetylneuraminic acid), fucose and/or N-acetylglucosamine with many and varied linkages between them. Almost all HMOs have a lactose moiety at their reducing end while sialic acid and/or fucose (when present) occupy terminal positions at the non-reducing ends. HMOs can be acidic (e.g. charged sialic acid containing oligosaccharides) or neutral (e.g. fucosylated oligosaccharides). See e.g. Chen, X., 2015. Advances in carbohydrate chemistry and biochemistry, 72, pp. 113-190.

HMOs found in human milk include Lacto-N-tetraose (LNT), Lacto-N-neotetraose (LNnT), 2′-Fucosyllactose (2′FL), 3-Fucosyllactose (3FL), Lactodifucotetraose (LDFT), Lacto-N-difucohexaose I (LNDFHI), Lacto-N-fucopentaose I (LNFP-I), Lacto-N-fucopentaose II (LNFP-II), Lacto-N-fucopentaose III (LNFP-III), Trifucosyllacto-N-hexaose (TF-LNH), 3′-Sialyllactose (3′SL), 6′-Sialyllactose (6′SL), Disialyllacto-N-tetraose (DSLNT), Sialyllacto-N-neotetraose c (LST c) and Fucosyldisialyllacto-N-hexaose I (FDS-LNH-I). See e.g. Soyyilmaz, B., et al., 2021. Nutrients, 13(8), p. 2737.

In some embodiments, the one or more HMOs comprise or consist of: (a) Lacto-N-difucohexaose I (LNDFHI); or (b) 2′-fucosyllactose (2′FL), 3-fucosyllactose (3FL), lactodifucotetraose (LDFT), lacto-N-fucopentaose II (LNFPII), lacto-N-fucopentaose I (LNFP-I), lacto-N-fucopentaose III (LNFP-III), lacto-N-difucohexaose I (LND-I), monofucosyl-lacto-N-hexaose III (MFLNH-III), lacto-N-fucopentaose V (LNFP-V), lacto-N-neodifucohexaose (LNnDFH) and lacto-N-fucopentaose VI (LNFP-VI).

The subject may be administered any suitable amounts of the one or more HMOs, in any suitable form and via any suitable route of administration (e.g. in any form and via any route described herein).

Suitable doses of HMOs are described in e.g. EFSA Panel on Nutrition, Novel Foods and Food Allergens (NDA), 2022. EFSA Journal, 20(5), p. e07257; EFSA Panel on Nutrition, Novel Foods and Food Allergens (NDA), 2021. EFSA Journal, 19(6), p. e06662; and EFSA Panel on Nutrition, Novel Foods and Food Allergens (NDA), 2022. EFSA Journal, 20(3), p. e07140.

Suitably, the one or more HMOs are administered to the subject in a total amount of at least about 0.5 g/day, at least about 1 g/day, or at least about 2 g/day. Suitably, the one or more HMOs are administered to the subject in an amount of about 10 g/day or less, about 8 g/day or less, or about 5 g/day or less. Suitably, the one or more HMOs are administered to the subject in an amount of from about 0.5 g/day to about 10 g/day, from about 1 g/day to about 8 g/day or from about 2 g/day to about 5 g/day.

The HMOs may be obtained by any suitable method. Suitable methods for synthesising HMOs will be well known to those of skill in the art. For example, processes have been developed for producing oligosaccharides by microbial fermentations, enzymatic processes, chemical syntheses, or combinations of these technologies (see e.g. Zeuner et al., 2019. Molecules, 24(11), p. 2033).

### Fucosylated HMOs

In some embodiments, the one or more HMOs comprise or consist of a combination of fucosylated HMOs. In some embodiments, the one or more HMOs consist of fucosylated HMOs. In some embodiments, the one or more HMOs are fucosylated HMOs.

As used herein, fucosylated HMOs may refer to neutral HMOs which contain one or more fucose sugar. Fucosylated HMOs may be monofucosylated, difucosylated, or trifucosylated and preferably do not contain sialic acid. Non-limiting example(s) of fucosylated HMOs include: 2′-fucosyllactose (2′FL); 3-fucosyllactose (3FL); lactodifucotetraose (LDFT); lacto-N-fucopentaose, such as lacto-N-fucopentaose I (LNFP-I), lacto-N-fucopentaose II (LNFP-II), lacto-N-fucopentaose III (LNFP-III) or lacto-N-fucopentaose V (LNFP-V); lacto-N-fucohexaose, such as lacto-N-difucohexaose I (LNDFH-I) and lacto-N-difucohexaose II (LNDFH-II); lacto-N-neofucopentaose V (LNnFP-V); lacto-N-neodifucosylhexaose (LNnDFH), such as lacto-N-neodifucosylhexaose II (LNnDFH-II); fucosyllacto-N-hexaose (FLNH), such as fucosyllacto-N-hexaose I (FLNH-I) and fucosyllacto-N-hexaose II (FLNH-II); fucosyllacto-N-neohexaose (F-LNnH), such as fucosyllacto-N-neohexaose I (F-LNnH-I) and fucosyllacto-N-neohexaose II (F-LNnH-II); monofucosyllacto-N-hexaose-III (MFNLH-III); difucosyllacto-N-hexaose (DF-LNH); difucosyllacto-N-neohexaose (DF-LNnH), such as difucosyllacto-N-neohexaose I (DF-LNnH-I) and difucosyllacto-N-neohexaose II (DF-LNnH-II); difucosyllacto-N-hexaose-a (DFLNHa); trifucosyllacto-N-hexaose (TF-LNH); fucosyl-para-lacto-N-hexaose (F-pLNH); difucosyl-para-lacto-N-hexaose (DF-pLNH); tr-fucosyl-para-lacto-N-hexaose (TF-pLNH); and combinations thereof. See e.g. Chen, X., 2015. Advances in carbohydrate chemistry and biochemistry, 72, pp. 113-190.

In some embodiments, the combination of fucosylated HMOs comprises or consists of lacto-N-difucohexaose I (LNDFHI), 2′-fucosyllactose (2′FL), 3-fucosyllactose (3FL), lactodifucotetraose (LDFT), and/or lacto-N-fucopentaose II (LNFPII). In some embodiments, the combination of fucosylated HMOs comprises or consists of lacto-N-difucohexaose I (LNDFHI), 2′-fucosyllactose (2′FL), 3-fucosyllactose (3FL), lactodifucotetraose (LDFT), lacto-N-fucopentaose II (LNFPII), lacto-N-fucopentaose I (LNFP-I), lacto-N-fucopentaose III (LNFP-III), lacto-N-difucohexaose I (LND-I), monofucosyl-lacto-N-hexaose III (MFLNH-III), lacto-N-fucopentaose V (LNFP-V), lacto-N-neodifucohexaose (LNnDFH) and lacto-N-fucopentaose VI (LNFP-VI).

In some embodiments, the combination of fucosylated HMOs comprises or consists of 2′-fucosyllactose (2′FL), 3-fucosyllactose (3FL), lactodifucotetraose (LDFT), lacto-N-fucopentaose II (LNFPII), lacto-N-fucopentaose I (LNFP-I), lacto-N-fucopentaose III (LNFP-III), lacto-N-difucohexaose I (LND-I), monofucosyl-lacto-N-hexaose III (MFLNH-III), lacto-N-fucopentaose V (LNFP-V), lacto-N-neodifucohexaose(LNnDFH) and/or lacto-N-fucopentaose VI (LNFP-VI). In some embodiments, the combination of fucosylated HMOs comprises or consists of 2′-fucosyllactose (2′FL), 3-fucosyllactose (3FL), lactodifucotetraose (LDFT), lacto-N-fucopentaose II (LNFPII), lacto-N-fucopentaose I (LNFP-I), lacto-N-fucopentaose III (LNFP-III), lacto-N-difucohexaose I (LND-I), monofucosyl-lacto-N-hexaose III (MFLNH-III), lacto-N-fucopentaose V (LNFP-V), lacto-N-neodifucohexaose (LNnDFH) and lacto-N-fucopentaose VI (LNFP-VI).

Suitably, the combination of fucosylated HMOs are administered to the subject in a total amount of at least about 0.5 g/day, at least about 1 g/day, or at least about 2 g/day. Suitably, the combination of fucosylated HMOs are administered to the subject in an amount of about 10 g/day or less, about 8 g/day or less, or about 5 g/day or less. Suitably, the combination of fucosylated HMOs are administered to the subject in an amount of from about 0.5 g/day to about 10 g/day, from about 1 g/day to about 8 g/day or from about 2 g/day to about 5 g/day.

### Lacto-N-Difucohexaose I (LNDFHI)

In some embodiments, the one or more HMOs comprise or consist of Lacto-N-difucohexaose I (LNDFHI). In some embodiments, the one or more HMOs consist of LNDFHI. In some embodiments, the one or more HMOs are LNDFHI.

Lacto-N-difucohexaose I (LNDFHI) is also been known as LNDFH-I or LNDI and is di-fucosylated, non-sialylated Galβ1-3GlcNAc core human-milk oligosaccharide that may have the following structure:

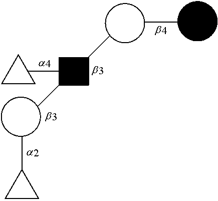

Where: [symbol] is fucose (Fuc), ◯ is galactose, ● is glucose, and ▪ is N-acetylglucosamine (GlcNAc)

Suitably, LNDFHI is administered to the subject in an amount of at least about 0.1 g/day, at least about 0.15 g/day, at least about 0.2 g/day, or at least about 0.5 g/day. Suitably, LNDFHI is administered to the subject in an amount of about 5 g/day or less, about 4 g/day or less, about 3 g/day or less, about 2 g/day or less, or about 1 g/day or less. Suitably, LNDFHI is administered to the subject in an amount of from about 0.1 g/day to about 5 g/day, from about 0.15 g/day to about 4 g/day, from about 0.2 g/day to about 3 g/day, or from about 0.5 g/day to about 2 g/day.

2′-Fucosyllactose (2′FL), 3-Fucosyllactose (3FL), Lactodifucotetraose (LDFT), Lacto-N-Fucopentaose II (LNFPII), Lacto-N-Fucopentaose I (LNFP-I), Lacto-N-Fucopentaose III (LNFP-III), Lacto-N-Difucohexaose I (LND-I), Monofucosyl-Lacto-N-Hexaose III (MFLNH-III), Lacto-N-Fucopentaose V (LNFP-V), Lacto-N-Neodifucohexaose (LNnDFH) and Lacto-N-Fucopentaose VI (LNFP-VI)

In some embodiments, the one or more HMOs comprise or consist of 2′-fucosyllactose (2′FL), 3-fucosyllactose (3FL), lactodifucotetraose (LDFT), lacto-N-fucopentaose II (LNFPII), lacto-N-fucopentaose I (LNFP-I), lacto-N-fucopentaose III (LNFP-III), lacto-N-difucohexaose I (LND-I), monofucosyl-lacto-N-hexaose III (MFLNH-III), lacto-N-fucopentaose V (LNFP-V), lacto-N-neodifucohexaose (LNnDFH) and lacto-N-fucopentaose VI (LNFP-VI). In some embodiments, the one or more HMOs consist of 2′FL, 3FL, LDFT, LNFPII, lacto-N-fucopentaose I (LNFP-I), lacto-N-fucopentaose III (LNFP-III), lacto-N-difucohexaose I (LND-I), monofucosyl-lacto-N-hexaose III (MFLNH-III), lacto-N-fucopentaose V (LNFP-V), lacto-N-neodifucohexaose (LNnDFH) and lacto-N-fucopentaose VI (LNFP-VI). In some embodiments, the one or more HMOs are 2′FL, 3FL, LDFT, LNFPII, lacto-N-fucopentaose I (LNFP-I), lacto-N-fucopentaose III (LNFP-III), lacto-N-difucohexaose I (LND-I), monofucosyl-lacto-N-hexaose III (MFLNH-III), lacto-N-fucopentaose V (LNFP-V), lacto-N-neodifucohexaose (LNnDFH) and lacto-N-fucopentaose VI (LNFP-VI).

2′-fucosyllactose (2′-FL) is a fucosylated, neutral trisaccharide composed of L-fucose, D-galactose, and D-glucose units. It is the most prevalent HMO naturally present in human breast milk, making up about 30% of all of HMOs and may have the following structure:

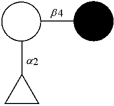

Where: [symbol] is fucose (Fuc), ◯ is galactose, and ● is glucose.

3-fucosyllactose (3-FL) is a fucosylated, neutral trisaccharide composed of L-fucose, D-galactose, and D-glucose units and may have the following structure:

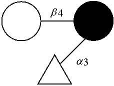

Where: [symbol] is fucose (Fuc), ◯ is galactose, and ● is glucose.

Lactodifucotetraose (LDFT) is also known as difucosyllactose (diFL or DFL) and may have the following structure:

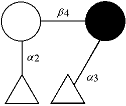

Where: [symbol] is fucose (Fuc), ◯ is galactose, and ● is glucose.

Lacto-N-fucopentaose II (LNFPII) is a fucosylated, non-sialylated Galβ1-3GlcNAc core (type 1 core) human milk oligosaccharide and may have the following structure:

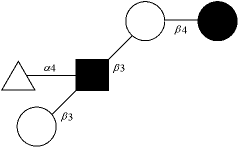

Where: [symbol] is fucose (Fuc), ◯ is galactose, ● is glucose, and ▪ is N-acetylglucosamine (GlcNAc)

The combination of 2′FL, 3FL, LDFT, LNFPII, LNFP-I, LNFP-III, LND-I, MFLNH-III, LNFP-V, LNnDFH and LNFP-VI may be administered separately, simultaneously or sequentially. In preferred embodiments, the combination of 2′FL, 3FL, LDFT, LNFPII, LNFP-I, LNFP-III, LND-I, MFLNH-III, LNFP-V, LNnDFH and LNFP-VI is administered simultaneously.

Suitably, 2′FL, 3FL, LDFT, LNFPII, LNFP-I, LNFP-III, LND-I, MFLNH-III, LNFP-V, LNnDFH and LNFP-VI are administered to the subject in a total amount of at least about 0.5 g/day, at least about 1 g/day, or at least about 2 g/day. Suitably, 2′FL, 3FL, LDFT, LNFPII, LNFP-I, LNFP-III, LND-I, MFLNH-III, LNFP-V, LNnDFH and LNFP-VI are administered to the subject in a total amount of about 10 g/day or less, about 8 g/day or less, or about 5 g/day or less. Suitably, 2′FL, 3FL, LDFT, LNFPII, LNFP-I, LNFP-III, LND-I, MFLNH-III, LNFP-V, LNnDFH and LNFP-VI are administered to the subject in a total amount of from about 0.5 g/day to about 10 g/day, from about 1 g/day to about 8 g/day or from about 2 g/day to about 5 g/day.

Suitably, 2′FL is administered to the subject in an amount of at least about 0.1 g/day, at least about 0.2 g/day, at least about 0.3 g/day, at least about 0.4 g/day, at least about 0.5 g/day, at least about 0.8 g/day, or at least about 1 g/day. Suitably, 2′FL is administered to the subject in an amount of about 5 g/day or less, about 4 g/day or less, or about 3 g/day or less. Suitably, 2′FL is administered to the subject in an amount of from about 0.1 g/day to about 5 g/day, from about 0.5 g/day to about 5 g/day, from about 0.8 g/day to about 4 g/day or from about 1 g/day to about 3 g/day.

Suitably, 3FL is administered to the subject in an amount of at least about 0.1 g/day, at least about 0.15 g/day, at least about 0.2 g/day, or at least about 0.5 g/day. Suitably, 3FL is administered to the subject in an amount of about 5 g/day or less, about 4 g/day or less, about 3 g/day or less, about 2 g/day or less, or about 1 g/day or less. Suitably, 3FL is administered to the subject in an amount of from about 0.1 g/day to about 5 g/day, from about 0.15 g/day to about 4 g/day, from about 0.2 g/day to about 3 g/day, or from about 0.5 g/day to about 2 g/day.

Suitably, LDFT is administered to the subject in an amount of at least about 0.1 g/day, at least about 0.15 g/day, or at least about 0.2 g/day. Suitably, LDFT is administered to the subject in an amount of about 5 g/day or less, about 1 g/day or less, about 0.8 g/day or less, or about 0.6 g/day or less. Suitably, LDFT is administered to the subject in an amount of from about 0.1 g/day to about 5 g/day, from about 0.1 g/day to about 1 g/day, from about 0.15 g/day to about 0.8 g/day or from about 0.2 g/day to about 0.6 g/day.

Suitably, LNFPII is administered to the subject in an amount of at least about 0.1 g/day, at least about 0.15 g/day, or at least about 0.2 g/day. Suitably, LNFPII is administered to the subject in an amount of about 5 g/day or less, about 1 g/day or less, about 0.8 g/day or less, or about 0.6 g/day or less. Suitably, LNFPII is administered to the subject in an amount of from about 0.1 g/day to about 5 g/day, from about 0.1 g/day to about 1 g/day, from about 0.15 g/day to about 0.8 g/day or from about 0.2 g/day to about 0.6 g/day.

Suitably, LNFP-1 is administered to the subject in an amount of at least about 0.1 g/day, at least about 0.15 g/day, or at least about 0.2 g/day. Suitably, LNFP-1 is administered to the subject in an amount of about 5 g/day or less, about 1 g/day or less, about 0.8 g/day or less, or about 0.6 g/day or less. Suitably, LNFP-1 is administered to the subject in an amount of from about 0.1 g/day to about 5 g/day, from about 0.1 g/day to about 1 g/day, from about 0.15 g/day to about 0.8 g/day or from about 0.2 g/day to about 0.6 g/day.

Suitably, LNFP-III is administered to the subject in an amount of at least about 0.1 g/day, at least about 0.15 g/day, or at least about 0.2 g/day. Suitably, LNFP-III is administered to the subject in an amount of about 5 g/day or less, about 1 g/day or less, about 0.8 g/day or less, or about 0.6 g/day or less. Suitably, LNFP-III is administered to the subject in an amount of from about 0.1 g/day to about 5 g/day, from about 0.1 g/day to about 1 g/day, from about 0.15 g/day to about 0.8 g/day or from about 0.2 g/day to about 0.6 g/day.

Suitably, LND-1 is administered to the subject in an amount of at least about 0.1 g/day, at least about 0.15 g/day, or at least about 0.2 g/day. Suitably, LND-1 is administered to the subject in an amount of about 5 g/day or less, about 1 g/day or less, about 0.8 g/day or less, or about 0.6 g/day or less. Suitably, LND-1 is administered to the subject in an amount of from about 0.1 g/day to about 5 g/day, from about 0.1 g/day to about 1 g/day, from about 0.15 g/day to about 0.8 g/day or from about 0.2 g/day to about 0.6 g/day.

Suitably, MFLNH-III is administered to the subject in an amount of at least about 0.1 g/day, at least about 0.15 g/day, or at least about 0.2 g/day. Suitably, MFLNH-III is administered to the subject in an amount of about 5 g/day or less, about 1 g/day or less, about 0.8 g/day or less, or about 0.6 g/day or less. Suitably, MFLNH-III is administered to the subject in an amount of from about 0.1 g/day to about 5 g/day, from about 0.1 g/day to about 1 g/day, from about 0.15 g/day to about 0.8 g/day or from about 0.2 g/day to about 0.6 g/day.

Suitably, LNFP-V is administered to the subject in an amount of at least about 0.1 g/day, at least about 0.15 g/day, or at least about 0.2 g/day. Suitably, LNFP-V is administered to the subject in an amount of about 5 g/day or less, about 1 g/day or less, about 0.8 g/day or less, or about 0.6 g/day or less. Suitably, LNFP-V is administered to the subject in an amount of from about 0.1 g/day to about 5 g/day, from about 0.1 g/day to about 1 g/day, from about 0.15 g/day to about 0.8 g/day or from about 0.2 g/day to about 0.6 g/day.

Suitably, LNnDFH is administered to the subject in an amount of at least about 0.1 g/day, at least about 0.15 g/day, or at least about 0.2 g/day. Suitably, LNnDFH is administered to the subject in an amount of about 5 g/day or less, about 1 g/day or less, about 0.8 g/day or less, or about 0.6 g/day or less. Suitably, LNnDFH is administered to the subject in an amount of from about 0.1 g/day to about 5 g/day, from about 0.1 g/day to about 1 g/day, from about 0.15 g/day to about 0.8 g/day or from about 0.2 g/day to about 0.6 g/day.

Suitably, LNFP-VI is administered to the subject in an amount of at least about 0.1 g/day, at least about 0.15 g/day, or at least about 0.2 g/day. Suitably, LNFP-VI is administered to the subject in an amount of about 5 g/day or less, about 1 g/day or less, about 0.8 g/day or less, or about 0.6 g/day or less. Suitably, LNFP-VI is administered to the subject in an amount of from about 0.1 g/day to about 5 g/day, from about 0.1 g/day to about 1 g/day, from about 0.15 g/day to about 0.8 g/day or from about 0.2 g/day to about 0.6 g/day.

### Nutritional Compositions

Suitably, the one or more HMOs are in the form of a composition. The composition may comprise the one or more HMOs in any effective amount.

In one aspect, the present invention provides a composition (e.g. a nutritional composition such as an infant formula, a follow-on formula, or a growing-up milk) comprising one or more human milk oligosaccharides (HMOs) for use in supporting behavioural development, improving social-emotional skills, reducing negative affectivity, and/or preventing an anxiety disorder and/or mood disorder.

In another aspect, the present invention provides use of one or more human milk oligosaccharides (HMOs) for the manufacture of a composition (e.g. a nutritional composition such as an infant formula, a follow-on formula, or a growing-up milk) for supporting behavioural development, improving social-emotional skills, reducing negative affectivity, and/or preventing an anxiety disorder and/or mood disorder.

In another aspect, the present invention provides a method for supporting behavioural development, improving social-emotional skills, reducing negative affectivity, and/or preventing an anxiety disorder and/or mood disorder, wherein the method comprises administering a composition (e.g. a nutritional composition such as an infant formula, a follow-on formula, or a growing-up milk) comprising one or more human milk oligosaccharides (HMOs).

The composition can be any type of composition in which the one or more HMOs can be incorporated, such as a composition in the form of a food or beverage product, a nutritional supplement, or a pharmaceutical composition. The composition may be in solid (e.g. powder), liquid or semi-liquid form. The one or more HMOs may be in the form of a food composition, a beverage, a nutritional formula, a nutritional supplement, a nutraceutical, or a pharmaceutical composition.

Food and beverage products include all products intended to be consumed orally by human beings, for the purpose of providing nutrition and/or pleasure. For example, the composition may be a food or beverage product, such as for infants, toddlers and/or young children. Examples of food and beverage products include dairy products such as milk products or yogurts, soups, sauces, sweet and savoury snacks, powdered drinks and cereal products.

In some embodiments, the one or more HMOs are in the form of a nutritional composition, a medical food product for clinical nutrition, or a supplement.

In some embodiments, the one or more HMOs are in the form of a nutritional composition. As used herein, a “nutritional composition” may mean a composition which nourishes a subject. This nutritional composition is usually to be taken orally or intravenously, and typically includes a lipid or fat source and a protein source.

The nutritional composition may be a synthetic nutritional composition. As used herein, a “synthetic nutritional composition” may mean a nutritional composition obtained by chemical and/or biological means that can be chemically substantially identical to a naturally composition.

In some embodiments, the one or more HMOs are in the form of a medical food product for clinical nutrition. As used herein, a “medical food product for clinical nutrition” may also be known as a “Food for Special Medical Purposes (FSMP)” and refer to specialised foods designed to help meet the nutritional or dietary needs of subjects living with a disease, disorder or medical condition who are temporarily or permanently unable to achieve an adequate nutritional intake from normal foods or through modification of the normal diet.

In some embodiments, the one or more HMOs are in the form of an infant formula, a follow-on formula, or a growing-up milk.

In some embodiments, the one or more HMOs are in the form of an infant formula. The term “infant formula” as used herein may refer to a foodstuff intended for particular nutritional use by infants during the first months of life and satisfying by itself the nutritional requirements of this category of person (see e.g. Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The infant formula can be a preterm infant formula, a human milk fortifier, a starter infant formula, a follow-on formula, a baby-food formula, or an infant cereal formula.

In some embodiments, the one or more HMOs are in the form of a starter infant formula. Typically, a “starter infant formula” is intended for infants from birth as a breast-milk substitute.

In some embodiments, the one or more HMOs are in the form of follow-on formula. A “follow-up formula” or “follow-on formula” may be given from the 6th month onwards. It may constitute the principal liquid element in the progressively diversified diet of this category of person.

In some embodiments, the one or more HMOs are in the form of a preterm infant formula. The term “preterm infant formula” as used herein means an infant formula intended for a preterm infant.

In some embodiments, the one or more HMOs are in the form of a milk fortifier. The term “milk fortifier” as used herein refers to liquid or solid nutritional compositions suitable for mixing with breast milk (which is human milk for a human milk fortifier) or infant formula. It is used to increase the calories, protein, minerals and vitamins in breast milk fed to preterm infants or infants with a low birth weight. The term “breast milk” is to be understood as the mother's milk or the colostrum of the mother or a donor's milk or the colostrum of a donor's milk.

In some embodiments, the one or more HMOs are in the form of a baby-food formula. The term “baby food formula” as used herein means a foodstuff intended for particular nutritional use by infants or children such as young children, during the first years of life.

In some embodiments, the one or more HMOs are in the form of a growing-up milk. The term “growing-up milk” (or GUM) as used herein refers to a milk formula product given from one year onwards. It is generally a dairy-based beverage adapted for the specific nutritional needs of young children (e.g. children aged from about 1 to about 3). Growing-up milk may also be known as “young-child formula” or “toddlers' milk”.

In some embodiments, the one or more HMOs are in the form of an infant cereal composition. The term “infant cereal composition” as used herein refers to a foodstuff intended for particular nutritional use by infants or children such as young children, during the first years of life.

In some embodiments, the one or more HMOs are in the form of a milk formula. The term “milk formula” as used herein may refer to a foodstuff intended for e.g. childhood nutrition, which may provide the sole source or supplemental nutrition for children e.g. aged about 3 years or older. In some embodiments, the milk formula is a grow milk.

In some embodiments, the composition is in a powder form and reconstituted in an aqueous medium (e.g. water) prior to administration. In other embodiments, the composition is in a liquid form ready for administration (e.g. a ready-to-feed formula).

In another embodiment, the one or more HMOs are in the form of a supplement. As used herein, a “supplement” or “dietary supplement” may be used to complement the nutrition of a subject (it is typically used as such but it might also be added to any kind of compositions intended to be ingested by the subject). When the composition is a supplement, it can be provided in the form of unit doses. Supplements are typically present in the form of a liquid, a gel, a powder or a tablet or capsule. Powder supplements typically encompass supplements to be dissolved in water or milk, or to be sprinkled on food or in a beverage. Such supplements are intended to provide additional nutrients and/or a health benefit to the subject consuming it. A supplement can be used for providing nutrients and/or a health benefit to human beings, as well as to animals.

In another embodiment, the one or more HMOs are in the form of a fortifier. The fortifier can be an infant formula fortifier or growing-up milk fortifier.

In another embodiment, the one or more HMOs are in the form of a pharmaceutical product. Pharmaceutical products include for example drops, syrups, powder, tablet or capsule products intended to treat or prevent an adverse medical condition in a subject in need thereof.

### Human Milk Oligosaccharides (HMOs)

The one or more HMOs may be present in the composition in any suitable amounts.

For example, in an infant formula, follow-on formula, or growing-up milk, the skilled person may base the amount of an HMO on the amounts found in human breast milk produced for an infant or child of the same age, in particular by a nutritionally replete mother (see e.g. Soyyilmaz, B., et al., 2021. Nutrients, 13(8), p. 2737). Such amounts may fall within the following ranges in human breast milk: LNDFHI: about 0.2 g/L to about 2.0 g/L; 2′FL: about 0.5 g/L to about 3.0 g/L; 3FL: about 0.2 g/L to about 2.0 g/L; LDFT: about 0.1 g/L to about 1.0 g/L; and LNFPII: about 0.1 g/L to about 1.0 g/L. However, they may be outside depending on for example bioavailability of said HMOs from infant formula in comparison to human breastmilk.

Suitably, the nutritional composition (e.g. infant formula, follow-on formula, or growing-up milk) comprises the one or more HMOs in a total amount of at least about 0.1 g/L, at least about 0.2 g/L, or at least about 0.5 g/L. Suitably, the nutritional composition (e.g. infant formula, follow-on formula, or growing-up milk) comprises the one or more HMOs in a total amount of about 10.0 g/L or less, about 8.0 g/L or less or about 5.0 g/L or less. Suitably, the nutritional composition (e.g. infant formula, follow-on formula, or growing-up milk) comprises the one or more HMOs in a total amount of from about 0.1 g/L to about 10.0 g/L, about 0.2 g/L to about 8.0 g/L, or about 0.5 g/L to about 5.0 g/L. If the nutritional composition is in the form of a powder these amounts may be following reconstitution with liquid according to the instructions provided.

Suitably, the nutritional composition (e.g. infant formula, follow-on formula, or growing-up milk) comprises the one or more HMOs in a total amount of at least about 0.5 wt %, at least about 1 wt %, or at least about 2 wt %, on a dry weight basis. Suitably, the nutritional composition (e.g. infant formula, follow-on formula, or growing-up milk) comprises the one or more HMOs in a total amount of about 10 wt % or less, about 8 wt % or less or about 5 wt % or less, on a dry weight basis. Suitably, the nutritional composition (e.g. infant formula, follow-on formula, or growing-up milk) comprises the one or more HMOs in a total amount of from about 0.5 wt % to about 10 wt %, about 1 wt % to about 8 wt %, or about 2 wt % to about 5 wt %, on a dry weight basis.

Suitably, the nutritional composition (e.g. infant formula, follow-on formula, or growing-up milk) comprises a combination of fucosylated HMOs in a total amount of at least about 0.1 g/L, at least about 0.2 g/L, or at least about 0.5 g/L. Suitably, the nutritional composition (e.g. infant formula, follow-on formula, or growing-up milk) comprises a combination of fucosylated HMOs in a total amount of about 10.0 g/L or less, about 8.0 g/L or less or about 5.0 g/L or less. Suitably, the nutritional composition (e.g. infant formula, follow-on formula, or growing-up milk) comprises a combination of fucosylated HMOs in a total amount of from about 0.1 g/L to about 10.0 g/L, about 0.2 g/L to about 8.0 g/L, or about 0.5 g/L to about 5.0 g/L. If the nutritional composition is in the form of a powder these amounts may be following reconstitution with liquid according to the instructions provided.

Suitably, the nutritional composition (e.g. infant formula, follow-on formula, or growing-up milk) comprises a combination of fucosylated HMOs in a total amount of at least about 0.5 wt %, at least about 1 wt %, or at least about 2 wt %, on a dry weight basis. Suitably, the nutritional composition (e.g. infant formula, follow-on formula, or growing-up milk) comprises a combination of fucosylated HMOs in a total amount of about 10 wt % or less, about 8 wt % or less or about 5 wt % or less, on a dry weight basis. Suitably, the nutritional composition (e.g. infant formula, follow-on formula, or growing-up milk) comprises a combination of fucosylated HMOs in a total amount of from about 0.5 wt % to about 10 wt %, about 1 wt % to about 8 wt %, or about 2 wt % to about 5 wt %, on a dry weight basis.

Suitably, the nutritional composition (e.g. infant formula, follow-on formula, or growing-up milk) comprises LNDFHI in an amount of at least about 0.1 g/L, at least about 0.15 g/L, or at least about 0.2 g/L. Suitably, the nutritional composition comprises LNDFHI in an amount of about 3.0 g/L or less, about 2.5 g/L or less, or about 2.0 g/L or less. Suitably, the nutritional composition (e.g. infant formula, follow-on formula, or growing-up milk) comprises LNDFHI in an amount of from about 0.1 g/L to about 3.0 g/L, from about 0.15 g/L to about 2.5 g/L, or from about 0.2 g/L to about 2.0 g/L. If the nutritional composition is in the form of a powder these amounts may be following reconstitution with liquid according to the instructions provided.

Suitably, the nutritional composition (e.g. infant formula, follow-on formula, or growing-up milk) comprises 2′FL in an amount of at least about 0.1 g/L, at least about 0.2 g/L, or at least about 0.5 g/L. Suitably, the nutritional composition comprises 2′FL in an amount of about 5.0 g/L or less, about 4.0 g/L or less, or about 3.0 g/L or less. Suitably, the nutritional composition (e.g. infant formula, follow-on formula, or growing-up milk) comprises 2′FL in an amount of from about 0.1 g/L to about 5.0 g/L, from about 0.2 g/L to about 4.0 g/L, or from about 0.5 g/L to about 3.0 g/L. If the nutritional composition is in the form of a powder these amounts may be following reconstitution with liquid according to the instructions provided.

Suitably, the nutritional composition (e.g. infant formula, follow-on formula, or growing-up milk) comprises 3FL in an amount of at least about 0.1 g/L, at least about 0.15 g/L, or at least about 0.2 g/L. Suitably, the nutritional composition comprises 3FL in an amount of about 3.0 g/L or less, about 2.5 g/L or less, or about 2.0 g/L or less. Suitably, the nutritional composition (e.g. infant formula, follow-on formula, or growing-up milk) comprises 3FL in an amount of from about 0.1 g/L to about 3.0 g/L, from about 0.15 g/L to about 2.5 g/L, or from about 0.2 g/L to about 2.0 g/L. If the nutritional composition is in the form of a powder these amounts may be following reconstitution with liquid according to the instructions provided.

Suitably, the nutritional composition (e.g. infant formula, follow-on formula, or growing-up milk) comprises LDFT in an amount of at least about 0.05 g/L, at least about 0.08 g/L, or at least about 0.1 g/L. Suitably, the nutritional composition comprises LDFT in an amount of about 2.0 g/L or less, about 1.5 g/L or less, or about 1.0 g/L or less. Suitably, the nutritional composition (e.g. infant formula, follow-on formula, or growing-up milk) comprises LDFT in an amount of from about 0.05 g/L to about 2.0 g/L, from about 0.08 g/L to about 1.5 g/L, or from about 0.1 g/L to about 1.0 g/L. If the nutritional composition is in the form of a powder these amounts may be following reconstitution with liquid according to the instructions provided.

Suitably, the nutritional composition (e.g. infant formula, follow-on formula, or growing-up milk) comprises LNFPII in an amount of at least about 0.05 g/L, at least about 0.08 g/L, or at least about 0.1 g/L. Suitably, the nutritional composition comprises LNFPII in an amount of about 2.0 g/L or less, about 1.5 g/L or less, or about 1.0 g/L or less. Suitably, the nutritional composition (e.g. infant formula, follow-on formula, or growing-up milk) comprises LNFPII in an amount of from about 0.05 g/L to about 2.0 g/L, from about 0.08 g/L to about 1.5 g/L, or from about 0.1 g/L to about 1.0 g/L. If the nutritional composition is in the form of a powder these amounts may be following reconstitution with liquid according to the instructions provided.

Suitably, the nutritional composition (e.g. infant formula, follow-on formula, or growing-up milk) comprises LNFP-I in an amount of at least about 0.05 g/L, at least about 0.08 g/L, or at least about 0.1 g/L. Suitably, the nutritional composition comprises LNFP-I in an amount of about 2.0 g/L or less, about 1.5 g/L or less, or about 1.0 g/L or less. Suitably, the nutritional composition (e.g. infant formula, follow-on formula, or growing-up milk) comprises LNFP-I in an amount of from about 0.05 g/L to about 2.0 g/L, from about 0.08 g/L to about 1.5 g/L, or from about 0.1 g/L to about 1.0 g/L. If the nutritional composition is in the form of a powder these amounts may be following reconstitution with liquid according to the instructions provided.

Suitably, the nutritional composition (e.g. infant formula, follow-on formula, or growing-up milk) comprises LNFP-III in an amount of at least about 0.05 g/L, at least about 0.08 g/L, or at least about 0.1 g/L. Suitably, the nutritional composition comprises LNFP-III in an amount of about 2.0 g/L or less, about 1.5 g/L or less, or about 1.0 g/L or less. Suitably, the nutritional composition (e.g. infant formula, follow-on formula, or growing-up milk) comprises LNFP-11l in an amount of from about 0.05 g/L to about 2.0 g/L, from about 0.08 g/L to about 1.5 g/L, or from about 0.1 g/L to about 1.0 g/L. If the nutritional composition is in the form of a powder these amounts may be following reconstitution with liquid according to the instructions provided.

Suitably, the nutritional composition (e.g. infant formula, follow-on formula, or growing-up milk) comprises LND-I in an amount of at least about 0.05 g/L, at least about 0.08 g/L, or at least about 0.1 g/L. Suitably, the nutritional composition comprises LND-I in an amount of about 2.0 g/L or less, about 1.5 g/L or less, or about 1.0 g/L or less. Suitably, the nutritional composition (e.g. infant formula, follow-on formula, or growing-up milk) comprises LND-I in an amount of from about 0.05 g/L to about 2.0 g/L, from about 0.08 g/L to about 1.5 g/L, or from about 0.1 g/L to about 1.0 g/L. If the nutritional composition is in the form of a powder these amounts may be following reconstitution with liquid according to the instructions provided.

Suitably, the nutritional composition (e.g. infant formula, follow-on formula, or growing-up milk) comprises MFLNH-III in an amount of at least about 0.05 g/L, at least about 0.08 g/L, or at least about 0.1 g/L. Suitably, the nutritional composition comprises MFLNH-III in an amount of about 2.0 g/L or less, about 1.5 g/L or less, or about 1.0 g/L or less. Suitably, the nutritional composition (e.g. infant formula, follow-on formula, or growing-up milk) comprises MFLNH-III in an amount of from about 0.05 g/L to about 2.0 g/L, from about 0.08 g/L to about 1.5 g/L, or from about 0.1 g/L to about 1.0 g/L. If the nutritional composition is in the form of a powder these amounts may be following reconstitution with liquid according to the instructions provided.

Suitably, the nutritional composition (e.g. infant formula, follow-on formula, or growing-up milk) comprises LNFP-V in an amount of at least about 0.05 g/L, at least about 0.08 g/L, or at least about 0.1 g/L. Suitably, the nutritional composition comprises LNFP-V in an amount of about 2.0 g/L or less, about 1.5 g/L or less, or about 1.0 g/L or less. Suitably, the nutritional composition (e.g. infant formula, follow-on formula, or growing-up milk) comprises LNFP-V in an amount of from about 0.05 g/L to about 2.0 g/L, from about 0.08 g/L to about 1.5 g/L, or from about 0.1 g/L to about 1.0 g/L. If the nutritional composition is in the form of a powder these amounts may be following reconstitution with liquid according to the instructions provided.

Suitably, the nutritional composition (e.g. infant formula, follow-on formula, or growing-up milk) comprises LNnDFH in an amount of at least about 0.05 g/L, at least about 0.08 g/L, or at least about 0.1 g/L. Suitably, the nutritional composition comprises LNnDFH in an amount of about 2.0 g/L or less, about 1.5 g/L or less, or about 1.0 g/L or less. Suitably, the nutritional composition (e.g. infant formula, follow-on formula, or growing-up milk) comprises LNnDFH in an amount of from about 0.05 g/L to about 2.0 g/L, from about 0.08 g/L to about 1.5 g/L, or from about 0.1 g/L to about 1.0 g/L. If the nutritional composition is in the form of a powder these amounts may be following reconstitution with liquid according to the instructions provided.

Suitably, the nutritional composition (e.g. infant formula, follow-on formula, or growing-up milk) comprises LNFP-VI in an amount of at least about 0.05 g/L, at least about 0.08 g/L, or at least about 0.1 g/L. Suitably, the nutritional composition comprises LNFP-VI in an amount of about 2.0 g/L or less, about 1.5 g/L or less, or about 1.0 g/L or less. Suitably, the nutritional composition (e.g. infant formula, follow-on formula, or growing-up milk) comprises LNFP-VI in an amount of from about 0.05 g/L to about 2.0 g/L, from about 0.08 g/L to about 1.5 g/L, or from about 0.1 g/L to about 1.0 g/L. If the nutritional composition is in the form of a powder these amounts may be following reconstitution with liquid according to the instructions provided.

### Other Components

A nutritional composition (e.g. infant formula, follow-on formula, or growing-up milk) of the invention may contain a protein source, a carbohydrate source and a lipid source. In some embodiments however, especially if a nutritional composition of the invention is a supplement or a fortifier, there may be only lipids (or a lipid source).

The nutritional composition (e.g. infant formula, follow-on formula, or growing-up milk) of the invention may comprise from about 50 kcal/100 mL to about 90 kcal/100 ml, from about 55 kcal/100 mL to about 85 kcal/100 ml, from about 60 kcal/100 mL to about 80 kcal/100 ml, from about 65 kcal/100 mL to about 75 kcal/100 ml, or from about 65 kcal/100 mL to about 70 kcal/100 ml. If the nutritional composition is in the form of a powder these amounts may be following reconstitution with liquid according to the instructions provided.

The nutritional composition (e.g. infant formula, follow-on formula, or growing-up milk) of the invention may comprise about 100 kcal/100 g to about 1000 kcal/100 g, about 200 kcal/100 g to about 800 kcal/100 g, or about 400 kcal/100 g to about 600 kcal/100 g, on a dry weight basis.

### Protein

A nutritional composition (e.g. infant formula, follow-on formula, or growing-up milk) according to the invention may contain a protein source. The protein may be in an amount of from about 1 g to about 4 g per 100 kcal, about 1.5 g to about 3 g per 100 kcal, or about 1.5 g to about 2.5 g per 100 kcal.

Protein sources based on, for example, whey, casein and mixtures thereof may be used as well as plant based protein sources, for example, based on soy. As far as whey proteins are concerned, the protein source may be based on acid whey or sweet whey or mixtures thereof and may include alpha-lactalbumin and beta-lactoglobulin in any desired proportions. In some embodiments the protein source is whey predominant (i.e. more than 50% of proteins are coming from whey proteins, such as more than 60% or more than 70%). The proteins may be intact or hydrolysed or a mixture of intact and hydrolysed proteins. By the term “intact” it is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component amino acids.

The proteins may be either fully or partially hydrolysed. If hydrolysed proteins are required, the hydrolysis process may be carried out as desired and as is known in the art. For example, whey protein hydrolysates may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source.

In one particular embodiment, the proteins of the composition are hydrolysed, fully hydrolysed or partially hydrolysed. The degree of hydrolysis (DH) of the protein can be between 2 and 20, or between 8 and 40, or between 20 and 60 or between 20 and 80 or more than 10, 20, 40, 60, 80 or 90.

At least 70%, 80%, 85%, 90%, 95% or 97% of the proteins may be hydrolysed. In a particular embodiment, 100% of the proteins are hydrolysed.

In one particular embodiment, the proteins of the composition are plant based protein.

### Carbohydrate

A nutritional composition (e.g. infant formula, follow-on formula, or growing-up milk) according to the present invention may contain a carbohydrate source. The carbohydrate may be in an amount of from about 5 g to about 20 g per 100 kcal, about 10 g to about 15 g per 100 kcal, about 8 g to about 15 g per 100 kcal.

Any carbohydrate source conventionally found in nutritional compositions such as lactose, sucrose, saccharose, maltodextrin, starch and mixtures thereof may be used although one of the preferred sources of carbohydrates is lactose.

### Lipid

A nutritional composition (e.g. infant formula, follow-on formula, or growing-up milk) according to the present invention may contain lipids and essential fatty acids. The lipids may be in an amount of from about 1 g to about 10 g per 100 kcal, about 2 g to about 8 g per 100 kcal, or about 3 g to about 8 g per 100 kcal.

Non-limiting examples of lipids include: palm olein, high oleic sunflower oil, high oleic safflower oil, canola oil, fish oil, coconut oil, bovine milk fat, and combinations thereof. It may be particularly beneficial if the composition comprises fat in an amount of about 25 to about 30 g/100 g dry weight of the composition. Non-limiting examples of essential fatty acids include: linoleic acid (LA), a-linolenic acid (ALA). Compositions of the invention may further contain gangliosides monosialoganglioside-3 (GM3) and disialogangliosides 3 (GD3), and combinations thereof.

### Others

A nutritional composition (e.g. infant formula, follow-on formula, or growing-up milk) of the invention may also contain all vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin A, vitamin 1, vitamin B2, vitamin B3, vitamin B6, vitamin B12, vitamin E, vitamin K1, vitamin K2, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population.

If necessary, a nutritional composition (e.g. infant formula, follow-on formula, or growing-up milk) of the invention may contain emulsifiers and stabilisers such as soy, lecithin, citric acid esters of mono- and diglycerides, and the like.

### Preparation of Compositions

The compositions according to the present invention may be prepared by any known or otherwise suitable manner. For example, a nutritional composition (e.g. infant formula, follow-on formula, or growing-up milk) may be proposed by blending together a source of protein with a carbohydrate source and a lipid source in appropriate proportions. If used, emulsifiers may be included at this stage. Vitamins and minerals may be added at this stage, but may also be added later to avoid thermal degradation. Water, preferably water which has been subjected to reverse osmosis or deionized water, may then be added and mixed in to form a liquid mixture. The temperature of mixing is preferably room temperature, but may also be higher. The liquid mixture may then be thermally treated to reduce bacterial loads. The mixture may then be homogenized.

If it is desired to produce a powdered composition, the homogenized mixture is dried in a suitable drying apparatus, such as a spray drier or freeze drier and converted into powder.

Processes used in the manufacture of formula are based on the concept that the products must be nutritionally adequate and microbiologically safe to consume. Thus, steps that eliminate or restrict microbiological growth are central to production processes. The processing technology for each specific formula is proprietary to the manufacturer but, in general, it involves the preservation of an oil-in-water (o/w) emulsion by dehydration in the case of powder products or, sterilization in the case of ready-to-feed or concentrated liquid products. Powdered formula may be produced using various processes, such as dry blending dehydrated ingredients to constitute a uniform formula or hydrating and wet-mixing a mixture of macro-ingredients, such as fat, protein and carbohydrate ingredients and then evaporating and spray drying the resultant mixture. A combination of the two processes described above may be used where a base powder is first produced by wet-mixing and spray drying all or some of the macro-ingredients and then dry blending the remaining ingredients, including carbohydrate, minerals and vitamins and other micronutrients, to create a final formula. Liquid formulae are available in a ready-to-feed format or as a concentrated liquid, which requires dilution, normally 1:1, with water. The manufacturing processes used for these products are similar to those used in the manufacture of recombined milk.

If it is desired to produce a liquid formula, the homogenized mixture may be filled into suitable containers, preferably aseptically. However, the liquid composition may also be retorted in the container, suitable apparatus for carrying out the filling and retorting of this nature is commercially available.

### Methods for Supporting Behavioural Development

The one or more HMOs may be used to promote behavioural development.

The terms “supporting”, “promoting”, “enhancing” or “improving” may be used interchangeably in the context of the present invention. They should be understood as comprising supporting or helping the normal development or growth of an individual. For example, the one or more HMOs may be used to improve behavioural skills.

As used herein, “behavioural development” may refer to a subject's development in terms of behavioural skills include communication, empathy and conflict resolution. In particular, the term “behavioural development” may refer to the development of social behaviour or social cognition, such as empathic and prosocial behaviour towards others, becoming able to cooperate with others, and learning more effectively from others (see e.g. Moore, C., Corbit K. 2019. Social cognition in infancy. Encyclopedia on Early Childhood Development). Suitable methods and parameters to determine behavioural development will be known to the skilled person (see e.g. Johnson, S. and Marlow, N., 2006. Early human development, 82(3), pp. 173-183).

As used herein, “supporting behavioural development” may refer to the support of normal behavioural development, for example during infancy, childhood and adolescence. Supporting behavioural development may result in normal behavioural skills.

In one aspect, the present invention provides one or more HMOs for use in supporting behavioural development in a subject. In another aspect, the present invention provides use of one or more HMOs for supporting behavioural development in a subject. In another aspect, the present invention provides a method for supporting behavioural development in a subject, comprising administering to the subject an effective amount of one or more HMOs.

In one aspect, the present invention provides a combination of fucosylated HMOs for use in supporting behavioural development in a subject. In another aspect, the present invention provides use of a combination of fucosylated HMOs for supporting behavioural development in a subject. In another aspect, the present invention provides a method for supporting behavioural development in a subject, comprising administering to the subject an effective amount of a combination of fucosylated HMOs.

In one aspect, the present invention provides LNDFHI for use in supporting behavioural development in a subject. In another aspect, the present invention provides use of LNDFHI for supporting behavioural development in a subject. In another aspect, the present invention provides a method for supporting behavioural development in a subject, comprising administering to the subject an effective amount of LNDFHI.

In one aspect, the present invention provides a combination of 2′FL, 3FL, LDFT, LNFPII, LNFP-I, LNFP-III, LND-I, MFLNH-III, LNFP-V, LNnDFH and LNFP-VI for use in supporting behavioural development in a subject. In another aspect, the present invention provides use of a combination of 2′FL, 3FL, LDFT, LNFPII, LNFP-I, LNFP-III, LND-I, MFLNH-III, LNFP-V, LNnDFH and LNFP-VI for supporting behavioural development in a subject. In another aspect, the present invention provides a method for supporting behavioural development in a subject, comprising administering to the subject an effective amount of a combination of 2′FL, 3FL, LDFT, LNFPII, LNFP-I, LNFP-III, LND-I, MFLNH-III, LNFP-V, LNnDFH and LNFP-VI.

### Methods for Improving Social-Emotional Skills

The one or more HMOs may be used to improve social-emotional skills.

In one aspect, the present invention provides one or more HMOs for use in improving social-emotional skills in a subject. In another aspect, the present invention provides use of one or more HMOs for improving social-emotional skills in a subject. In another aspect, the present invention provides a method for improving social-emotional skills in a subject, comprising administering to the subject an effective amount of one or more HMOs.

In one aspect, the present invention provides a combination of fucosylated HMOs for use in improving social-emotional skills in a subject. In another aspect, the present invention provides use of a combination of fucosylated HMOs for improving social-emotional skills in a subject. In another aspect, the present invention provides a method for improving social-emotional skills in a subject, comprising administering to the subject an effective amount of a combination of fucosylated HMOs.

In one aspect, the present invention provides LNDFHI for use in improving social-emotional skills in a subject. In another aspect, the present invention provides use of LNDFHI for improving social-emotional skills in a subject. In another aspect, the present invention provides a method for improving social-emotional skills in a subject, comprising administering to the subject an effective amount of LNDFHI.

In one aspect, the present invention provides a combination of 2′FL, 3FL, LDFT, LNFPII, LNFP-I, LNFP-III, LND-I, MFLNH-III, LNFP-V, LNnDFH and LNFP-VI for use in improving social-emotional skills in a subject. In another aspect, the present invention provides use of a combination of 2′FL, 3FL, LDFT, LNFPII, LNFP-I, LNFP-III, LND-I, MFLNH-III, LNFP-V, LNnDFH and LNFP-VI for improving social-emotional skills in a subject. In another aspect, the present invention provides a method for improving social-emotional skills in a subject, comprising administering to the subject an effective amount of a combination of 2′FL, 3FL, LDFT, LNFPII, LNFP-I, LNFP-III, LND-I, MFLNH-III, LNFP-V, LNnDFH and LNFP-VI.

As used herein, “improving social-emotional skills” may refer to the support of normal social-emotional skills, for example during infancy, childhood and adolescence. Supporting social-emotional skills may result in normal social-emotional skills. Within the context of the present invention, the expressions “social-emotional” or “social-emotional development” may relate to social-emotional skills that develop from birth onwards allowing for increasing autonomy, self-regulation, and relationships. Social-emotional development can be assessed and monitored behaviourally using clinical interviews, direct behaviour observations, and rating systems as well as parent- and teacher-report questionnaires.

In some embodiments, social-emotional skills are determined by an Infant Behavior Questionnaire (IBQ), such as the IBQ-Revised (IBQ-R). The “IBQ-R (revised Infant Behavior Questionnaire) questionnaire” is a widely used parent-report measure for assessing dimensions of temperament along 14 scales, as reported below, in infants between 3 and 12 months of age (see e.g. Gartstein, M. A. and Rothbart, M. K., 2003. Infant behavior and development, 26(1), pp. 64-86).

| IBQ-R scale | Scale description |
|---|---|
| Approach | Rapid approach, excitement, and positive anticipation of |
| | pleasurable activities. Amount of vocalization exhibited by the |
| | baby in daily activities. |
| Vocal Reactivity | Pleasure and enjoyment related to high stimulus intensity, rate, |
| | complexity, novelty, and incongruity. Smiling and laugher during |
| | general caretaking and play. |
| Activity level | Gross motor activity, including movement of arms and legs, |
| | squirming and locomotor activity. |
| Perceptual Sensitivity | Detection of slight, low intensity stimuli from the external |
| | environment. |
| Sadness | Lowered mood and activity related to personal suffering, physical |
| | state, object loss, or inability to perform desired action; general |
| | low mood. |
| Distress to Limitations | Fussing, crying or showing distress while (a) in a confining place |
| | or position; (b) in caretaking activities; (c) unable to perform a |
| | desired action. |
| Fear | Startle or distress to sudden changes in stimulation, novel |
| | physical objects or social stimuli; inhibited approach to novelty. |
| Falling Reactivity/rate of | Rate of recovery from peak distress, excitement, or general |
| recovery from distress | arousal; ease of falling asleep. |
| Low intensity pleasure | Amount of pleasure or enjoyment related to low stimulus |
| | intensity, rate complexity, novelty and incongruity. |
| High intensity pleasure | Pleasure and enjoyment related to high stimuli intensity, rate, |
| | complexity, novelty and incongruity |
| Cuddliness | Expression of enjoyment and molding of the body being held by |
| | a caregiver. |
| Duration of Orienting | Attention to and/or interaction with a single object for extended |
| | period of time. |
| Soothability | Reduction of fussing, crying, or distress, when soothing |
| | techniques are used by the caregiver. |
| Social Fear | Slow/inhibited approach and/or distress in social situations that |
| | involve novelty/uncertainty. |
| Attentional Shifting | The ability to transfer attentional focus from one activity/task to |
| | another. |

In some embodiments, social-emotional skills are determined by the Toddler Behavior Assessment Questionnaire (TBAQ). The “Toddler Behavior Assessment Questionnaire (TBAQ)” is used to measure behaviours in young children between 16 to 36 months (Goldsmith, H. H., 1996. Child development, 67(1), pp. 218-235). The TBAQ contains 120 items and 11 scales: activity level, perceptual sensitivity, inhibitory control, soothability, appropriate attentional allocation, sadness, object fearfulness, anger, social fearfulness, pleasure and interest. Items are rated on a 7-point scale ranging from 1=never to 4=about half the time to 7=always during the past month.

The one or more HMOs may be used to improve social skills. In one aspect, the present invention provides one or more HMOs for use in improving social skills in a subject. In another aspect, the present invention provides use of one or more HMOs for improving social skills in a subject. In another aspect, the present invention provides a method for improving social skills in a subject, comprising administering to the subject an effective amount of one or more HMOs.

Within the context of the present invention, “social skill” may encompass any competence or behaviour observed in a social situation or environment and/or facilitating interaction and communication with others, both verbally and non-verbally, and development of social skills starts in the first few months of life. For example, infants begin to show signs of emotions, such as joy, anger, sadness, interest, fear, disgust and surprise as distinct facial expression.

Infants also begin to laugh out and begin to socialize with people through babbling. Social skills continue developing in toddlers and children, including for example to begin imitating the environments around 15 months, clinging to caretakers in new situation (social fearfulness) at 18 months, parallel playing with other children at around 24 months, etc.

The one or more HMOs may be used to improve emotional skills. In one aspect, the present invention provides one or more HMOs for use in improving emotional skills in a subject. In another aspect, the present invention provides use of one or more HMOs for improving emotional skills in a subject. In another aspect, the present invention provides a method for improving emotional skills in a subject, comprising administering to the subject an effective amount of one or more HMOs.

Within the context of the present invention, “emotional skill” may refer to the capacity to experience and express personal feelings, as well as recognize and interpret those of others. Emotional skills are linked for example to mood, pleasure, sadness. The development of emotional skills occurs in conjunction with neural, cognitive and behavioural development from birth throughout childhood, adolescence and adulthood.

The one or more HMOs according to the present invention may improve social-emotional skills of a subject at 9, 12, 18, 24 or 36 months of age. The one or more HMOs according to the present invention may improve social-emotional skills of a subject aged between 9 to 36 months. The one or more HMOs according to the present invention may improve social-emotional skills of a subject of 9 months of age.

### Methods for Reducing Negative Affectivity

The one or more HMOs may be used to reduce negative affectivity.

In one aspect, the present invention provides one or more HMOs for use in reducing negative affectivity in a subject. In another aspect, the present invention provides use of one or more HMOs for reducing negative affectivity in a subject. In another aspect, the present invention provides a method for reducing negative affectivity in a subject, comprising administering to the subject an effective amount of one or more HMOs.

In one aspect, the present invention provides a combination of fucosylated HMOs for use in reducing negative affectivity in a subject. In another aspect, the present invention provides use of a combination of fucosylated HMOs for reducing negative affectivity in a subject. In another aspect, the present invention provides a method for reducing negative affectivity in a subject, comprising administering to the subject an effective amount of a combination of fucosylated HMOs.

In one aspect, the present invention provides LNDFHI for use in reducing negative affectivity in a subject. In another aspect, the present invention provides use of LNDFHI for reducing negative affectivity in a subject. In another aspect, the present invention provides a method for reducing negative affectivity in a subject, comprising administering to the subject an effective amount of LNDFHI.

In one aspect, the present invention provides a combination of 2′FL, 3FL, LDFT, LNFPII, LNFP-I, LNFP-III, LND-I, MFLNH-III, LNFP-V, LNnDFH and LNFP-VI for use in reducing negative affectivity in a subject. In another aspect, the present invention provides use of a combination of 2′FL, 3FL, LDFT, LNFPII, LNFP-I, LNFP-III, LND-I, MFLNH-III, LNFP-V, LNnDFH and LNFP-VI for reducing negative affectivity in a subject. In another aspect, the present invention provides a method for reducing negative affectivity in a subject, comprising administering to the subject an effective amount of a combination of 2′FL, 3FL, LDFT, LNFPII, LNFP-I, LNFP-III, LND-I, MFLNH-III, LNFP-V, LNnDFH and LNFP-VI.

As used herein, “negative affectivity” may refer to a personality variable that involves the experience of negative emotions and poor self-concept. Negative affectivity subsumes a broad range of negative mood states, including fear, anxiety, hostility, scorn, and disgust (see e.g. Watson, D., et al., 1988. Journal of abnormal psychology, 97(3), p. 346). Suitably, in the context of the present invention, the term “negative affectivity” may be used interchangeably with the term “negative emotions” or “negative mood states”.

The subject's negative affectivity can be determined by any suitable method. In some embodiments, the subject's negative affectivity is determined by an Infant Behavior Questionnaire (IBQ), for example the IBQ-Revised (IBQ-R). For example, negative affectivity may be a domain constructed out of the following scales on the IBQ-R: sadness, fear, distress to limitations, activity level, and falling reactivity. The one or more HMOs according to the present invention may reduce sadness, fear, and/or distress to limitations. The one or more HMOs according to the present invention may improve activity level and/or rate of recovery from distress.

The one or more HMOs according to the present invention may reduce negative affectivity of a subject at 9, 12, 18, 24 or 36 months of age. The one or more HMOs according to the present invention may reduce negative affectivity of a subject aged between 9 to 36 months. The one or more HMOs according to the present invention may reduce negative affectivity of a subject of 9 months of age.

The one or more HMOs may promote mood and joy behaviour. Within the context of the present invention, the term “mood” may refer to an emotional state of an individual at a particular time. Within the context of the present invention, the term “joy” may refer to a feeling of great pleasure and happiness. Joy is a powerful antidote to stress which has negative effects during infant and child development. Ongoing stressful events trigger feelings of anxiety. Promoting joy may lessen anxious feelings. Promoting mood and joy behaviours in an infant may reduce the risk of experiencing mood difficulties that may lead to aggressive or disruptive behaviours, to loneliness and may cause behavioural problems later in life. The one or more HMOs may promote high intensity pleasure, in particular pleasure and enjoyment related to high stimulus intensity, rate, complexity, novelty and incongruity, and reduces sadness, in particular reducing lowered mood and activity related to suffering physical state, object loss or inability to perform an action in an infant.

The one or more HMOs according to the present invention may promote mood and joy behaviour of a subject at 9, 12, 18, 24 or 36 months of age. The one or more HMOs according to the present invention may promote mood and joy behaviour of a subject aged between 9 to 36 months. The one or more HMOs according to the present invention may promote mood and joy behaviour of a subject of 9 months of age.

### Methods for Preventing Anxiety Disorder or Mood Disorders

The one or more HMOs may be used to prevent an anxiety disorder and/or mood disorder.

In one aspect, the present invention provides one or more HMOs for use in preventing an anxiety disorder and/or mood disorder in a subject. In another aspect, the present invention provides use of one or more HMOs for preventing an anxiety disorder and/or mood disorder in a subject. In another aspect, the present invention provides a method for preventing an anxiety disorder and/or mood disorder in a subject, comprising administering to the subject an effective amount of one or more HMOs.

In one aspect, the present invention provides a combination of fucosylated HMOs for use in preventing an anxiety disorder and/or mood disorder in a subject. In another aspect, the present invention provides use of a combination of fucosylated HMOs for preventing an anxiety disorder and/or mood disorder in a subject. In another aspect, the present invention provides a method for preventing an anxiety disorder and/or mood disorder in a subject, comprising administering to the subject an effective amount of a combination of fucosylated HMOs.

In one aspect, the present invention provides LNDFHI for use in preventing an anxiety disorder and/or mood disorder in a subject. In another aspect, the present invention provides use of LNDFHI for preventing an anxiety disorder and/or mood disorder in a subject. In another aspect, the present invention provides a method for preventing an anxiety disorder and/or mood disorder in a subject, comprising administering to the subject an effective amount of LNDFHI.

In one aspect, the present invention provides a combination of 2′FL, 3FL, LDFT LNFPII, LNFP-I, LNFP-III, LND-I, MFLNH-III, LNFP-V, LNnDFH and LNFP-VI for use in preventing an anxiety disorder and/or mood disorder in a subject. In another aspect, the present invention provides use of a combination of 2′FL, 3FL, LDFT, LNFPII, LNFP-I, LNFP-III, LND-I, MFLNH-III, LNFP-V, LNnDFH and LNFP-VI for preventing an anxiety disorder and/or mood disorder in a subject. In another aspect, the present invention provides a method for preventing an anxiety disorder and/or mood disorder in a subject, comprising administering to the subject an effective amount of a combination of 2′FL, 3FL, LDFT, LNFPII, LNFP-I, LNFP-III, LND-I, MFLNH-III, LNFP-V, LNnDFH and LNFP-VI.

Within the context of the present invention, “preventing” a disorder may refer to decrease of the severity of the disorder or relieve of the symptoms of induced by the disorder, such as stress and/or decrease of complications caused by the disorder. Within the context of the present invention, “preventing” a disorder may also refer to reducing the risk of the disorder occurring.

The one or more HMOs may improve behaviours in social interactions, for example play, and/or in response to novelty and/or new situations, and/or with new people and/or strangers in an infant older than 12 months, a toddler or a young child. Improving social-emotional skills in an infant, a toddler and/or a young child may reduce the risk of developing behavioural problems later in life, for example anxiety disorder or depressive disorders (see e.g. Watson, D., et al., 1988. Journal of abnormal psychology, 97(3), p. 346), as it is believed that lack of social skills can lead in poor outcomes in child development such as hindering child's ability to create relationship, affecting child's adjustment at school, leading to loneliness and may cause behavioural problems later in life.

Examples of anxiety disorders include generalized anxiety disorder, social anxiety disorder (social phobia) and separation disorder. Generalized anxiety disorder, separation anxiety disorder, and social phobia are thought to be among the most common, earliest-onset childhood psychiatric disorders and depression in adulthood and for continuing comorbidity and impaired quality of life throughout the lifespan (see e.g. Copeland, W. E., et al., 2009. Archives of general psychiatry, 66(7), pp. 764-772).

Mood disorders are a cluster of mental disorders that include for example bipolar and depressive disorders. Mood is defined as a pervasive and sustained feeling tone that is endured internally and which impacts nearly all aspects of a person's behaviour in the external world. Mood disorders are described by marked disruptions in emotions (severe lows called depression or highs called hypermania or mania). These include bipolar disorder, cyclothymia, hypomania, major depressive disorder, disruptive mood dysregulation, persistent depressive disorder, and premenstrual dysphoric disorder.

### Method for Supporting Gross Motor Skills and Visual Reception

The one or more HMOs may be used to support gross motors skills and visual reception.

In some aspect, the present invention provides 3FL for use in supporting gross motor skills and visual reception in a subject. In another aspect, the present invention provides use of 3FL for supporting gross motor skills and visual reception in a subject. In another aspect, the present invention provides a method for supporting gross motor skills and visual reception in a subject, comprising administering to the subject an effective amount of 3FL.

In some embodiments, gross motor skills and visual perception are determined using the Mullen Scales of Early Learning scores (MSEL). MSEL is a validated, widely used infant cognitive development assessment tool, comprising five domains: fine motor, gross motor, visual reception, receptive language, and expressive language (Mullen E M. Mullen scales of early learning. AGS Circle Pines, MN; 1995).

### Subject

The subject may be any suitable subject. Suitably, the subject may be a human.

In preferred embodiments, the subject is an infant, a toddler, or a young child. The term “infant” may refer to a subject aged from about 0 years to about 1 year. The term “toddler” may refer to a subject aged from about 1 year to about 3 years. The term “young child” may refer to a subject aged from about 3 years to about 10 years, from about 3 years to about 9 years, from about 3 years to about 8 years, from about 3 years to about 7 years, from about 3 years to about 6 years, or from about 3 years to about 5 years.

In preferred embodiments, the subject is an infant. In some embodiments, the subject is a newborn infant. In some embodiments, the subject is a preterm infant.

In some embodiments, the subject is aged about 5 years or less, about 4 years or less, about 3 years or less, about 2 years or less, or about 1 year or less. In some embodiments, the subject is aged about 12 months or less, about 11 months or less, about 10 months or less, about 9 months or less, about 8 months or less, about 7 months or less, or about 6 months or less. In some embodiments, the subject is aged about 9 months or less.

In some embodiments, the one or more HMOs are administered during the first 1, 3, 6, 9, 12, 18, 14, or 36 months of life of a subject. For example, the one or more HMOs are administered to a subject starting from birth up to 12, 18, 24 or 36 months.

The subject may be at risk of inadequate behavioural development. Major risks for poor child development include intrauterine growth restriction, stunting, iodine deficiency, iron-deficiency anemia, malaria, lead exposure, HIV, maternal depression, and inadequate cognitive stimulation. Additionally, maternal education and breastfeeding have been identified as protective factors (Walker, S. P., et al., 2011. The lancet, 378(9799), pp. 1325-1338). In some embodiments, the subject has and/or has suffered from intrauterine growth restriction, stunting, iodine deficiency, iron-deficiency anemia, malaria, lead exposure, HIV, maternal depression, and/or inadequate cognitive stimulation. The subject may be at risk of inadequate social-emotional skills. The subject may be at risk of negative affectivity.

The subject may have suffered from and/or may be suffering from inadequate behavioural development. Any suitable screening tools may be used (see e.g. Johnson, S. and Marlow, N., 2006. Early human development, 82(3), pp. 173-183). The subject may have suffered from and/or may be suffering from inadequate social-emotional skills. The subject may have suffered from and/or may be suffering from negative affectivity. This may be determined by any method known in the art, for example using an Infant Behaviour Questionnaire (IBQ), such as the IBQ-Revised (IBQ-R), see e.g. Gartstein, M. A. and Rothbart, M. K., 2003. Infant behavior and development, 26(1), pp. 64-86.

### EXAMPLES

The invention is further described with reference to the following examples. It will be appreciated that the invention as claimed is not intended to be limited in any way by these examples.

### Example 1—Association of Behavioural Outcomes with Breast Milk Concentrations of Human Milk Oligosaccharides (HMOs)

The inventors investigated the association of behavioural outcomes with breast milk concentrations of specific human milk oligosaccharides (HMOs) or with composite scores representative of specific groups of HMOs in human breast milk-fed infants.

A total of 195 infants were tested. Breastmilk HMO levels were tested in breastmilk collected at two months and three months. Infant behaviours were assessed with an Infant Behaviour Questionnaire-Revised (IBQ-R). A regression model approach was used where the covariates included: maternal net income, SES/education of mothers, weight, gestational age, gender of the infant (covariates were imputed, missing values are replaced by the population mean). Negative affectivity domain was constructed out of several scales from the IBQR (which measures temperament of the infant and is self-reported by the mother) including sadness, fear, distress to limitations, activity level, and falling reactivity.

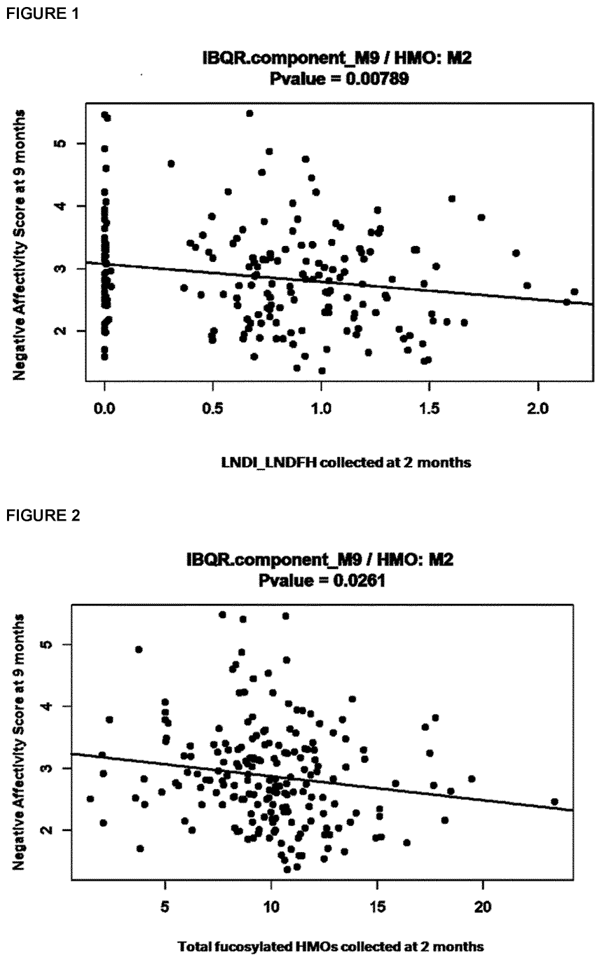

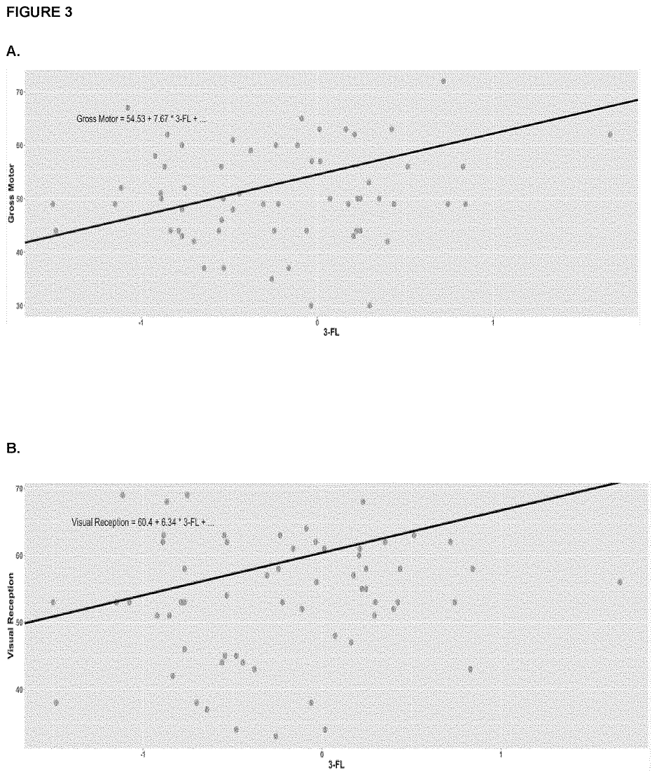

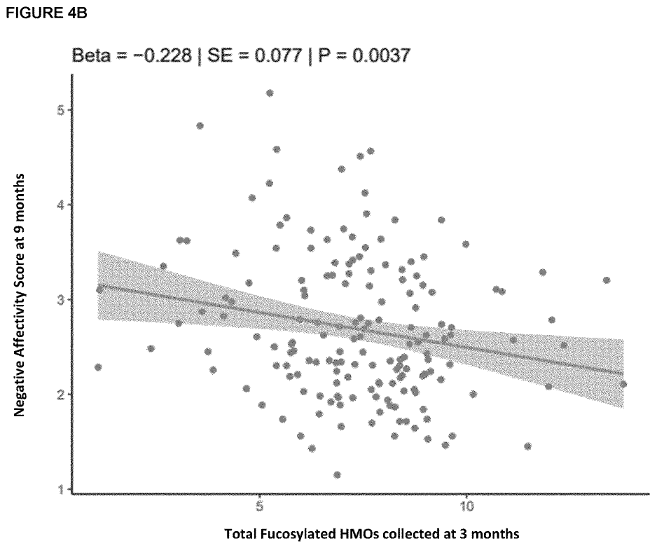

Specific fucosylated HMOs (LNDFHI, and 2′FL, 3FL, LDFT, LNFPII, LNFP-I, LNFP-III, LND-I, MFLNH-III, LNFP-V, LNnDFH and LNFP-VI) are associated with reduction in negative emotions in infants at 9 months (see Table 1 below, FIG. 1, FIG. 2, FIG. 4 A and B).

TABLE 1
Results of regression model approach assessing association
between behavioural outcomes and HMOs

| | | | | When |
|---|---|---|---|---|
| HMOs | IBQR | P | N | Collected |
| LNDFHI | Reduced | 0.0079 | 195 | 2 Months |
| | negative | | | |
| | affectivity | | | |
| 2′FL, 3FL, LDFT, LNFPII, | Reduced | 0.026 | 195 | 2 Months |
| LNFP-I, LNFP-III, LND-I, | negative | | | |
| MFLNH-III, LNFP-V, | affectivity | | | |
| LNnDFH and LNFP-VI | | | | |
| LNDFHI, 2′FL, 3FL, LDFT, | Reduced | 0.0056 | 195 | 2 Months |
| LNFPII, LNFP-I, LNFP-III, | negative | | | |
| LND-I, MFLNH-III, LNFP- | affectivity | | | |
| V, LNnDFH and LNFP-VI | | | | |
| LNDFHI, 2′FL, 3FL, LDFT, | Reduced | 0.0037 | 195 | 3 months |
| LNFPII, LNFP-I, LNFP-III, | negative | | | |
| LND-I, MFLNH-III, LNFP- | affectivity | | | |
| V, LNnDFH and LNFP-VI | | | | |

### Example 2—Association of Motor Gross Skills and Visual Reception with Breast Milk Concentrations of Human Milk Oligosaccharides (HMOs)

The inventors investigated the association of motor gross skills and visual reception with breast milk concentrations of specific human milk oligosaccharides (HMOs).

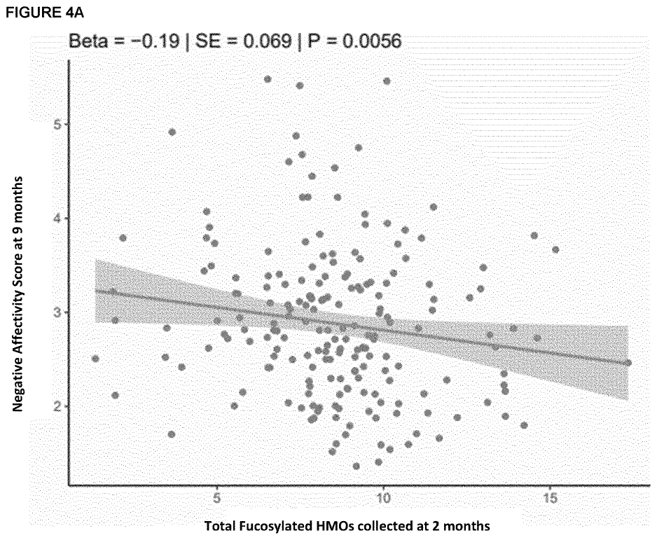

A total of 105 infants between 2 weeks and 12 months were assessed in the Mullen Scales of Early Learning (MSEL), and their mother's milk were collected for HMOs analyses. Of the 105 infants, 60 visited once while the remaining subjects had at most four visits, leading to a total of 170 MSEL assessments and breastmilk samples. Breastmilk was collected using a standard procedure at each visit. MSEL assessed between 3 and 24 months of age. Association between BM levels of 3FL was evaluated using linear regression model. Due to the cohort design, we had to remove the potential confounding effects from age using smooth spline regression prior to conducting association analyses (Hastie T J et al. Generalized additive models, Boca Raton, Florida, USA. CRC Press; 1990) with the MSEL scores in all 5 subscales, of which A significantly positive association were observed between 3FL for visual reception (p=0.041, ES=6.34) and gross motor skills (p=0.027, ES=7.67) (see FIG. 3).

### EMBODIMENTS

Various preferred features and embodiments of the present invention will now be described with reference to the following numbered paragraphs (paras).

1. One or more human milk oligosaccharides (HMOs) for use in supporting behavioural development in a subject, wherein the one or more HMOs comprise or consist of: (a) lacto-N-difucohexaose I (LNDFHI); and/or (b) 2′-fucosyllactose (2′FL), 3-fucosyllactose (3FL), lactodifucotetraose (LDFT) and lacto-N-fucopentaose II (LNFPII).

2. Use of one or more human milk oligosaccharides (HMOs) for supporting behavioural development in a subject, wherein the one or more HMOs comprise or consist of: (a) lacto-N-difucohexaose I (LNDFHI); and/or (b) 2′-fucosyllactose (2′FL), 3-fucosyllactose (3FL), lactodifucotetraose (LDFT) and lacto-N-fucopentaose II (LNFPII).

3. A method for supporting behavioural development in a subject, comprising administering to the subject an effective amount of one or more human milk oligosaccharides (HMOs), wherein the one or more HMOs comprise or consist of: (a) lacto-N-difucohexaose I (LNDFHI); and/or (b) 2′-fucosyllactose (2′FL), 3-fucosyllactose (3FL), lactodifucotetraose (LDFT) and lacto-N-fucopentaose II (LNFPII).

4. The one or more HMOs for use according to para 1, the use according to para 2, or the method according to para 3, wherein the one or more HMOs comprise or consist of LNDFHI.

5. The one or more HMOs for use according to para 1 or 4, the use according to para 2 or 4, or the method according to para 3 or 4, wherein the one or more HMOs comprise or consist of 2′FL, 3FL, LDFT and LNFPII.

6. The one or more HMOs for use according to any of paras 1 or 4-5, the use according to any of paras 2 or 4-5, or the method according to any of paras 3 or 4-5, wherein the one or more HMOs are administered by oral administration.

7. The one or more HMOs for use according to any of paras 1 or 4-6, the use according to any of paras 2 or 4-6, or the method according to any of paras 3 or 4-6, wherein the one or more HMOs are administered separately, simultaneously or sequentially, preferably wherein the one or more HMOs are administered simultaneously.

8. The one or more HMOs for use according to any of paras 1 or 4-7, the use according to any of paras 2 or 4-7, or the method according to any of paras 3 or 4-7, wherein the one or more HMOs are administered to the subject in a total amount of from about 0.5 g/day to about 10 g/day.

9. The one or more HMOs for use according to any of paras 1 or 4-8, the use according to any of paras 2 or 4-8, or the method according to any of paras 3 or 4-8, wherein LNDFHI is administered to the subject in an amount of from about 0.1 g/day to about 5 g/day.

10. The one or more HMOs for use according to any of paras 1 or 4-9, the use according to any of paras 2 or 4-9, or the method according to any of paras 3 or 4-9, wherein: (i) 2′FL is administered to the subject in an amount of from about 0.1 g/day to about 5 g/day; (ii) 3FL is administered to the subject in an amount of from about 0.1 g/day to about 5 g/day; (iii) LDFT is administered to the subject in an amount of from about 0.1 g/day to about 5 g/day; and/or (iv) LNFPII is administered to the subject in an amount of from about 0.1 g/day to about 5 g/day.

11. The one or more HMOs for use according to any of paras 1 or 4-10, the use according to any of paras 2 or 4-10, or the method according to any of paras 3 or 4-10, wherein the one or more HMOs are in the form of a nutritional composition.

12. The one or more HMOs for use according to para 11, the use according to para 11, or the method according to para 11, wherein the nutritional composition is an infant formula, a follow-on formula, or a growing-up milk, preferably wherein the nutritional composition is an infant formula.

13. The one or more HMOs for use according to para 11 or 12, the use according to para 11 or 12, or the method according to para 11 or 12, wherein the nutritional composition comprises the one or more HMOs in a total amount of from about 0.5 g/L to about 10 g/L.

14. The one or more HMOs for use according to any of paras 11-13, the use according to any of paras 11-13, or the method according to any of paras 11-13, wherein the nutritional composition comprises LNDFHI in an amount of from about 0.2 g/L to about 2.0 g/L.

15. The one or more HMOs for use according to any of paras 11-14, the use according to any of paras 11-14, or the method according to any of paras 11-14, wherein the nutritional composition comprises: (i) 2′FL in an amount of from about 0.5 g/L to about 3.0 g/L; (ii) 3FL in an amount of from about 0.2 g/L to about 2.0 g/L; (iii) LDFT in an amount of from about 0.05 g/L to about 0.5 g/L; and/or (iv) LNFPII in an amount of from about 0.05 g/L to about 0.5 g/L.

16. The one or more HMOs for use according to any of paras 1 or 4-15, the use according to any of paras 2 or 4-15, or the method according to any of paras 3 or 4-15, wherein the subject is a human subject.

17. The one or more HMOs for use according to any of paras 1 or 4-16, the use according to any of paras 2 or 4-16, or the method according to any of paras 3 or 4-16, wherein the subject is an infant, a toddler or a young child.

18. The one or more HMOs for use according to any of paras 1 or 4-17, the use according to any of paras 2 or 4-17, or the method according to any of paras 3 or 4-17, wherein the subject is an infant.

19. The one or more HMOs for use according to any of paras 1 or 4-18, the use according to any of paras 2 or 4-18, or the method according to any of paras 3 or 4-18, wherein the subject is aged about 12 months or younger, about 11 months or younger, about 10 months or younger, or about 9 months or younger.

20. The one or more HMOs for use according to any of paras 1 or 4-19, the use according to any of paras 2 or 4-19, or the method according to any of paras 3 or 4-19, wherein the one or more HMOs improve the subject's social-emotional skills.

21. The one or more HMOs for use according to any of paras 1 or 4-20, the use according to any of paras 2 or 4-20, or the method according to any of paras 3 or 4-20, wherein the one or more HMOs reduce the subject's negative affectivity.

22. The one or more HMOs for use according to para 21, the use according to para 21, or the method according to para 21, wherein the subject's negative affectivity is determined by an Infant Behavior Questionnaire (IBQ), preferably the IBQ-Revised (IBQ-R).

23. Use of one or more HMOs for improving social-emotional skills in a subject, wherein the one or more HMOs comprise or consist of: (a) lacto-N-difucohexaose I (LNDFHI); and/or (b) 2′-fucosyllactose (2′FL), 3-fucosyllactose (3FL), lactodifucotetraose (LDFT) and lacto-N-fucopentaose II (LNFPII).

24. Use of one or more HMOs for reducing negative affectivity in a subject, wherein the one or more HMOs comprise or consist of: (a) lacto-N-difucohexaose I (LNDFHI); and/or (b) 2′-fucosyllactose (2′FL), 3-fucosyllactose (3FL), lactodifucotetraose (LDFT) and lacto-N-fucopentaose II (LNFPII).

25. The use according to para 23 or 24, wherein the one or more HMOs comprise or consist of LNDFHI.

26. The use according to any of paras 23-25, wherein the one or more HMOs comprise or consist of 2′FL, 3FL, LDFT and LNFPII.

27. The use according to any of paras 23-26, wherein the one or more HMOs are administered by oral administration.

28. The use according to any of paras 23-27, wherein the one or more HMOs are administered separately, simultaneously or sequentially, preferably wherein the one or more HMOs are administered simultaneously.

29. The use according to any of paras 23-28, wherein the one or more HMOs are administered to the subject in a total amount of from about 0.5 g/day to about 10 g/day.

30. The use according to any of paras 23-29, wherein LNDFHI is administered to the subject in an amount of from about 0.1 g/day to about 5 g/day.

31. The use according to any of paras 23-30, wherein: (i) 2′FL is administered to the subject in an amount of from about 0.1 g/day to about 5 g/day; (ii) 3FL is administered to the subject in an amount of from about 0.1 g/day to about 5 g/day; (iii) LDFT is administered to the subject in an amount of from about 0.1 g/day to about 5 g/day; and/or (iv) LNFPII is administered to the subject in an amount of from about 0.1 g/day to about 5 g/day.

32. The use according to any of paras 23-31, wherein the one or more HMOs are in the form of a nutritional composition.

33. The use according to para 32, wherein the nutritional composition is an infant formula, a follow-on formula, or a growing-up milk, preferably wherein the nutritional composition is an infant formula.

34. The use according to para 32 or 33, wherein the nutritional composition comprises the one or more HMOs in a total amount of from about 0.5 g/L to about 10 g/L.

35. The use according to any of paras 32-34, wherein the nutritional composition comprises LNDFHI in an amount of from about 0.2 g/L to about 2.0 g/L.

36. The use according to any of paras 32-35, wherein the nutritional composition comprises: (i) 2′FL in an amount of from about 0.5 g/L to about 3.0 g/L; (ii) 3FL in an amount of from about 0.2 g/L to about 2.0 g/L; (iii) LDFT in an amount of from about 0.05 g/L to about 0.5 g/L; and/or (iv) LNFPII in an amount of from about 0.05 g/L to about 0.5 g/L.

37. The use according to any of paras 23-36, wherein the subject is a human subject.

38. The use according to any of paras 23-37, wherein the subject is an infant, a toddler or a young child.

39. The use according to any of paras 23-38, wherein the subject is an infant.

40. The use according to any of paras 23-39, wherein the subject is aged about 12 months or younger, about 11 months or younger, about 10 months or younger, or about 9 months or younger.

41. The one or more HMOs for use according to any of paras 1 or 4-19, the use according to any of paras 2 or 4-19, or the method according to any of paras 3 or 4-19, wherein the one or more HMOs improve the subject's gross motors skills and/or visual reception.

42. The one or more HMOs for use according to para 41, the use according to 41, or the method according to para 41, wherein the one or more HMOs comprises or consists of 3FL.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

1. A method of supporting behavioural development in a subject, the method comprising administering one or more human milk oligosaccharides (HMOs) to the subject,
wherein the one or more HMOs comprise at least one of: lacto-N-difucohexaose I (LNDFHI), 2′-fucosyllactose (2′FL), 3-fucosyllactose (3FL), lactodifucotetraose (LDFT), lacto-N-fucopentaose II (LNFPII), lacto-N-fucopentaose I (LNFP-I), lacto-N-fucopentaose III (LNFP-III), lacto-N-difucohexaose I (LND-I), monofucosyl-lacto-N-hexaose III (MFLNH-III), lacto-N-fucopentaose V (LNFP-V), lacto-N-neodifucohexaose (LNnDFH) and lacto-N-fucopentaose VI (LNFP-VI).

2. The method according to claim 1, wherein the one or more HMOs comprise LNDFHI.

3. The method according to claim 1, wherein the one or more HMOs comprise 2′FL, 3FL, LDFT, LNFPII, LNFP-I, LNFP-III, LND-I, MFLNH-III, LNFP-V, LNnDFH and LNFP-VI.

4. The method according to claim 1, wherein the one or more HMOs are administered by oral administration.

5. The method according to claim 1, wherein the one or more HMOs are administered separately, simultaneously or sequentially.

6. The method according to claim 1, wherein the one or more HMOs are administered to the subject in a total amount from about 0.5 g/day to about 10 g/day.

7. The method according to claim 1, wherein the one or more HMOs are administered in a nutritional composition.

8. The method according to claim 7, wherein the nutritional composition is an infant formula, a follow-on formula, or a growing-up milk.

9. The method according to claim 7, wherein the nutritional composition comprises the one or more HMOs in a total amount from about 0.5 g/L to about 10 g/L.

10. The method according to claim 1, wherein the subject is an infant, a toddler or a young child.

11. The method according to claim 1, wherein the subject is aged about 12 months or younger.

12. The method according to claim 1, wherein the one or more HMOs are administered in an amount effective to improve the subject's social-emotional skills.

13. The method according to claim 1, wherein the one or more HMOs are administered in an amount effective to reduce the subject's negative affectivity.

14. (canceled)

15. (canceled)

16. The method according to claim 1, wherein the one or more HMOs are administered simultaneously.

17. The method according to claim 7, wherein the nutritional composition is an infant formula.

18. The method according to claim 1, wherein the subject is an infant.

19. The method according to claim 1, wherein the subject is aged about 9 months or younger.

20. The method according to claim 13, wherein the subject's negative affectivity is determined by the Infant Behavior Questionnaire Revised (IBQ-R).
